(19) United States
(12) Patent Application Publication
(10) Pub. No.: US 2006/0105357 A1
(43) Pub. Date: May 18, 2006

(54) Tissue sensor and uses thereof

(75) Inventors: Frank Benesch, Cambridge, MA (US); Victoria Margit Barbata, Providence, RI (US); Robert Francis Valentini, Cranston, RI (US); Herman H. Vandenburgh, Providence, RI (US); Gregory Philip Crawford, Barrington, RI (US)

Correspondence Address: PALMER & DODGE, LLP;KATHLEEN M. WILLIAMS, 111 HUNTINGTON AVENUE, BOSTON, MA, 02199, US

(73) Assignee: Myomics, Inc.

(21) Appl. No.: 11/051,736

(22) Filed: Feb. 4, 2005

Related U.S. Application Data
(63) Continuation-in-part of application No. 10/241,618, filed on Sep. 11, 2002.
Application No. 10/241,618 is a continuation of application No. 09/252,324, filed on Feb. 18, 1999, now abandoned.
(60) Provisional application No. 60/075,054, filed on Feb. 18, 1998.
(60) Provisional application No. 60/086,370, filed on May 22, 1998.

(51) Int. Cl. C12Q 1/68; G01N 1/30; C12M 1/34

(52) U.S. Cl. 435/6; 435/40.5; 435/287.2

(57) ABSTRACT

Described are assemblies for screening a compound for bioactivity, the assemblies comprising a tissue and a sensor. A change in a biological parameter is measured by the sensor, such that a change in a parameter occurring when the tissue is contacted with a candidate compound is detected by the sensor. Assemblies provided herein include single sensor/tissue assemblies and arrays of such assemblies, including plates comprising tissues in combination with one or more sensors. Also provided are methods of screening a compound using tissue/sensor tissue assemblies as described.

This application is a Continuation In Part of U.S. patent application Ser. No. 10/241,618, filed Sep. 11, 2002, which is a Continuation of U.S. patent application Ser. No. 09/252,324, filed Feb. 18, 1999, now abandoned, which claims the priority of U.S. Provisional application No. 60/075,054, filed Feb. 18, 1998 and U.S. Provisional application No. 60/086,370, filed May 22, 1998.

### FIELD OF THE INVENTION

The invention relates to the measurement of a parameter of a tissue and to measurement of bioactivity of a compound on such a tissue.

### BACKGROUND OF THE INVENTION

In vitro screening of compounds for biological activity has been disclosed in the prior art as assays, for example, in which monolayers of tissue cultured cells are exposed to a candidate compound and a biological response in the cells is measured. For example, monolayers of disorganized muscle fibers have been shown to respond to anabolic growth factors. See Vandenburgh et al. (Vandenburgh et al., Am. J. Physiol. 260: C475-C484, 1991) which discloses induction of hypertrophy of skeletal muscle myofibers by insulin and insulin-like growth factors. See Janeczko et al. (Janeczko et al., J. Biol. Chem. 259: 6292-6297, 1984) which discloses that multiplication-stimulating activity inhibits intracellular proteolysis in muscle monolayer cultures. See Vandenburgh et al. (Vandenburgh et al., Am. J. Physiol. 259: C232-C240, 1990) which discloses modulation of protein degradation and synthesis by prostaglandins in muscle monolayer cultures. In vivo methods of compound screening also have been performed in animals to test the biological response of a host tissue (Dupont et al., J. Appl. Physiol. 80: 734-741, 1996).

Most in vitro testing is performed with continuous cell lines which do not retain the properties of the original organ from which they were derived. In addition most cell lines are useful for only several days. Tissue-cultured cells of primary tissue have also been utilized for testing of compounds in vitro. Such primary cell cultures also have relatively short-term viability in vitro (about 7-14 days) in the differentiated state (Volz et al., J. Mol. Cell. Cardiol. 23, 161-173, 1991). Most cell types in a two-dimensional, monolayer culture system (e.g. skeletal muscle, cardiac muscle, fibroblasts, bone and cartilage) dedifferentiate within about 14 days. In addition, certain cell types (e.g. muscle, fibroblasts, bone and cartilage) are anchorage dependent, and when these adherent cells grown as a monolayer are spontaneously released into the culture medium, they will die.

### SUMMARY OF THE INVENTION

Described herein are methods and compositions applicable tot he measurement of a parameter of a tissue and to the measurement of bioactivity of a compound on such tissue.

In one aspect, disclosed herein is a composition comprising a container comprising at least one viable tissue in combination with a sensor.

In one embodiment, the tissue is independent of the sensor. In another embodiment, the tissue is not independent from the sensor.

In another embodiment, the tissue comprises muscle cells. In another embodiment, the muscle cells are smooth, skeletal or cardiac muscle cells. Other tissues include, as non-limiting examples, ligament, tendon or other connective tissues. It is contemplated that additional tissues can include, for example, liver (which can be useful for monitoring toxicity), nerve, pancreas, etc. Cells, extracellular matrix, growth factors and other components necessary to generate, for example, liver, nerve and pancreas tissues in vitro are known in the art.

In another embodiment, the tissue is organized.

In another embodiment, the sensor measures an optical, physical, chemical or electrical property of the tissue. In another embodiment, the sensor measures at least one of muscle contraction, muscle relaxation, muscle hypertrophy, muscle atrophy, and muscle length or diameter.

In another embodiment, the device further comprises a device to provide a readout for a change in a property of the tissue.

In another aspect, described herein is a plate comprising at least one tissue in combination with a sensor.

In one embodiment, the sensor is independent from the tissue. In another embodiment, the tissue is not independent from the sensor.

In another embodiment, the plate comprises at least two microposts, e.g., 2, 3, 4, 10, 12, 20, 24, 48, 50, 96, 100, 192, 200, 384, 400, 500, 768, 800, 1000, 2000, 5000, etc. In one embodiment, the microposts are attached to the plate. In another embodiment, the microposts are supported by an extracellular matrix material. In one embodiment, the extracellular matrix material comprises collagen.

In another embodiment, the tissue is in contact with two or more microposts. In another embodiment, the tissue is in contact with and located between at least two of the microposts.

Also described is an array of microposts associated with tissue.

In one embodiment, the array comprises one or more lattice unit cells defined by the arrangement of the microposts.

In another embodiment, the plate comprises a plurality of wells that comprise the tissue. In one embodiment, the wells are anisotropic or shaped so as to encourage the formation of anisotropic tissue. In one embodiment, a well of the plurality of wells comprises at least two microposts. In another embodiment, a well of the plurality of wells comprises an array of microposts. In another embodiment, the array comprises one or more lattice unit cells defined by the arrangement of the microposts. In another embodiment, the tissue is in contact with and located between at least two of the microposts. In another embodiment, the tissue is in contact with and located between a plurality of pairs of the microposts in the array.

In another embodiment, the tissue is in contact with and located between each micropost defining a lattice unit cell.

In another embodiment, the plate comprises muscle cells. In another embodiment, the muscle cells are selected from smooth, skeletal or cardiac muscle cells.

In another embodiment, the tissue is organized.

In another embodiment, the plate comprises one or more essentially linear grooves.

In another embodiment, the one or more essentially linear grooves are located in one or more wells on the plate. In another embodiment, the grooves are arranged parallel to each other.

In another embodiment, the tissue is arranged in the one or more grooves.

In another embodiment, one or more of the grooves comprises at least two microposts. In another embodiment, tissue is in contact with and located between at least two of the microposts.

In another embodiment, the sensor measures a change in the distance between the microposts.

In another embodiment, the sensor measures at least one of muscle contraction, muscle relaxation, muscle hypertrophy, muscle atrophy and muscle length/diameter.

In another embodiment, the plate is associated with or comprises a device to provide a readout for a change in a property of the tissue.

In another aspect, described herein is an array comprising at least one tissue in combination with a sensor. The sensor can be independent from the tissue or not independent from the tissue.

In one embodiment, the tissue comprises muscle cells. In another embodiment, the muscle cells can be smooth, skeletal or cardiac muscle cells.

In one embodiment, the tissue is organized. In another embodiment, the tissue can be unorganized.

In another embodiment, the sensor is optical, physical, electrical, or chemical. In another embodiment, the sensor measures at least one of muscle contraction, muscle relaxation, muscle hypertrophy, muscle atrophy and muscle length/diameter.

In another embodiment, the assembly further comprises or is in communication with a device to provide a readout for a change in a property of the tissue.

In another embodiment, the array comprises a plurality of microposts.

In another embodiment, the tissue is in contact with and located between at least two of the microposts. The tissue can be under tension between the microposts.

In another embodiment, the array further comprises or is associated with a device to provide a readout for a change in a property of the tissue.

In another aspect, described herein is an apparatus comprising at least a tissue in combination with: a) a sensor; and b) a device that provides a readout for a change in a property of the tissue.

Also described herein is a method of screening a compound for bioactivity, comprising contacting a candidate bioactive compound with a tissue, wherein the tissue is in combination with a sensor, and measuring in the tissue a biological parameter that is associated with bioactivity, wherein a change in the biological parameter that occurs as a result of the contacting step is indicative of bioactivity of the candidate compound.

Also described herein is a method of screening a library of compounds for bioactivity, comprising contacting a candidate bioactive compound from the library with a tissue, wherein the tissue is in combination with a sensor, and measuring in a tissue a biological parameter that is associated with bioactivity, wherein a change in the biological parameter that occurs as a result of the contacting step is indicative of bioactivity of the candidate compound.

Also described herein is a method of identifying a compound that increases or decreases muscle contraction or muscle relaxation comprising contacting a candidate compound with a tissue, wherein the tissue is in combination with a sensor, and measuring in the tissue, muscle contraction, wherein an increase or decrease in muscle contraction that occurs as a result of the contacting step is indicative of the compound modulating muscle contraction.

In each of the screening methods described herein: the sensor can be independent or not independent of the tissue; the tissue can comprise muscle cells, e.g., smooth, skeletal or cardiac muscle cells; the tissue can be organized; the sensor can measure an optical, physical, chemical, genetic or electrical property of the tissue; the sensor may measure at least one of muscle contraction, muscle relaxation, muscle hypertrophy and muscle length; and the tissue/sensor combination can further comprise a device to provide a readout for a change in a property of the tissue.

In another aspect, provided herein is a method of monitoring the effect of an agent on a tissue, the method comprising the steps of: a) providing a plurality of tissues formed in vitro, wherein at least one of the tissues is in combination with a sensor; b) contacting the plurality of tissues with an agent; c) obtaining a measurement from the sensor; and d) detecting a nucleic acid sequence in one of the tissues, wherein an effect of the agent on the tissues is determined.

In one embodiment, the step of detecting a nucleic acid sequence in a tissue comprises isolating nucleic acid from a tissue of the plurality.

In another embodiment, the step of detecting a nucleic acid sequence in a tissue comprises amplification of a nucleic acid sequence from a tissue of the plurality.

In another embodiment, the step of detecting a nucleic acid sequence a tissue comprises hybridization of nucleic acid prepared from the tissue to an array.

In another embodiment, the step of detecting a nucleic acid sequence in a tissue comprises obtaining a genetic expression profile for the tissue.

In another embodiment, the contacting step is repeated at least once.

In another embodiment, steps (c) and (d) are repeated at least once. In a further embodiment, the steps of detecting detect a change in the genetic expression profile for the tissues.

In another embodiment, the tissue is prepared from cells from an individual having a condition affecting said tissue.

In another embodiment, the tissue comprises a genetically modified cell.

Also described herein is a method of inducing muscle contraction or muscle relaxation in a tissue in combination with a sensor, wherein the tissue is contacted with a compound, a mechanical force and/or an electrical force.

Also described herein is a method of measuring permeability of a compound that increases or decreases at least one of muscle contraction, muscle relaxation, muscle hypertrophy, muscle mass and muscle length, comprising introducing the compound into a sensor, wherein the sensor is in combination with a tissue, and wherein the permeability is measured by determining a change in at least one of muscle contraction, muscle hypertrophy, muscle mass and muscle length of the tissue.

Compounds identified using the methods described herein can be used, for example, to treat or correct a structural or genetic defect, e.g., that causing muscular dystrophy or other disease.

In each of the methods described herein, the sensor can be independent from the tissue or not independent from the tissue. Further, the tissue can comprise muscle cells, e.g., smooth, skeletal or cardiac muscle cells.

The tissue can be organized.

In one embodiment, the sensor is optical, physical, electrical or chemical.

In another embodiment, the biological parameter is selected from the group consisting of: muscle contraction, muscle relaxation, muscle hypertrophy, muscle length, gene expression, mRNA expression, protein expression, enzymatic activity.

In another embodiment, the method is performed in real-time.

In another aspect, described herein is a device for measuring a parameter of a tissue, the device comprising: a) a hollow tube; b) a distal end of elastic material extending from the hollow tube; c) a tissue adhered to an exterior surface of the distal end.

In one embodiment, the distal end is approximately spherical.

In another embodiment, the tube communicates with a pressure transducer.

In another embodiment, a change in pressure inside the tube is detected by the pressure transducer.

In another embodiment, the tissue is grown on the exterior surface of the distal end.

In another embodiment, the tissue comprises muscle tissue, e.g., cardiac muscle, smooth muscle or striated muscle. In another embodiment, contraction of the muscle tissue results in a detectable change in pressure inside the tube.

In another embodiment, the elastic material comprises an elastomeric (eg silicon, polyurethane etc) membrane.

In another embodiment, there is an array of devices as described above.

In another aspect, described herein is a method of determining a compound's effect on a tissue, the method comprising contacting a device as described above with the compound and detecting a change in pressure inside the tube. In one embodiment, the tissue comprises muscle.

In another aspect, described herein is a device comprising: a) a hollow tube; and b) an elastic membrane covering or stretched over a distal end of the tube, the membrane in contact with a tissue.

In one embodiment, the tube communicates with a pressure transducer.

In another embodiment, the membrane comprises a silicon membrane.

In another embodiment, the tissue is grown on the membrane.

In another embodiment, tissue is not grown on the membrane.

In another embodiment, the tissue comprises muscle tissue, e.g., cardiac muscle, smooth muscle or striated muscle.

In another embodiment, contraction of the muscle tissue results in a detectable change in pressure inside the tube.

Also provided is an array of devices as described above.

In another aspect, there is described a method of determining a compound's effect on a tissue, the method comprising contacting a device as described above with the compound, and detecting a change in pressure inside the tube. In one embodiment, the tissue comprises muscle.

In another aspect, a tissue/sensor combination described herein can be used in combination with methods that measure gene activity to correlate parameters measured by the sensor with changes in gene activity. For example, a plurality of similar or identical tissues can be prepared and monitored for an activity, e.g., muscle contraction, in response to a drug or stimulus as described herein. Individual tissues from the plurality (or parts of them) can be harvested at various times during the course of drug or stimulus application and used to analyze the expression of one or more genes in the tissue. Collection of data, both directly from the sensors or indirectly from further harvesting of tissues, can continue over time on remaining tissues after such harvest, provided enough tissues are prepared. The arrays described herein, including, but not limited to arrays of tissues prepared in individual wells or in tubs within wells, including, for example, micropost arrays, are well suited for such methods.

Combining the data obtained through the sensor with gene expression data can provide powerful insights into the activity of known or new drug agents on such tissues. Gene activity can be monitored by, for example, PCR targeting one or a number of genes, known or unknown. In one aspect, nucleic acid derived from such tissues can be used to probe a microarray, thereby providing a genetic expression profile for that time point. Other approaches to genetic profiling, e.g., approaches based on differential display or similar methods are known in the art. By obtaining simultaneous genetic expression data, the pathways influenced by a given drug or stimulus that affects mechanical function can also be identified. The data obtained by monitoring a number of similar or identical tissues for a parameter such as muscle contraction, relaxation, etc., over time using a tissue/sensor combination as described herein can also be combined with data regarding protein expression profile or proteomic analysis in a similar fashion.

In part because of the number of tissues that can be simultaneously or at least contemporaneously monitored, as well as because tissues described herein can be maintained for extended periods of time, the tissue/sensor combinations described herein are well suited for long term studies, e.g., on the order of days, weeks, or even months. As such, they can provide data regarding tissue function in response to a drug or stimulus and ways in which the response or the tissue can change with long-term or repeat exposure to the drug or stimulus. This can be predictive of, for example, the long term effects of a drug or stimulus on the organization or function of a tissue. Such long term studies can also identify, for example, activities of known drugs on tissues which may not become apparent in shorter term studies. Thus, the tissue-sensor combinations described herein can permit the identification of new beneficial uses of known drugs, e.g., where a drug or compound known to be tolerated in vivo is found to have an activity not previously appreciated. Such long term studies can also potentially identify previously unappreciated harmful effects of known or new drugs. This long-term predictive aspect becomes even more powerful when coupled with the ability to monitor the genetic or proteomic profile of tissues from the same experiment at times corresponding to the mechanical or physical measurements provided by a sensor.

The invention also features a kit comprising a plurality of organized tissues wherein each organized tissue is contained in a container.

In a preferred embodiment of the kit the container comprises a culture plate containing a plurality of tubs, wherein each tub contains a tissue or a plurality of tissues in medium and under conditions wherein the tissue is viable, long-term. The tubs can be isolated from other tubs, as, for example, separate wells in a multi-well culture plate, or, alternatively, a plurality of tubs can be present in a single well. In either instance, the tubs can be arranged in an array, thereby facilitating more rapid gathering of information regarding the effect(s) of a compound or compounds.

Additional kits can include, for example, a kit comprising one or more a drum sensor assemblies as described herein and one or more tissues, or one or more bubble-type tissue/sensor assemblies. Further included in such kits can be, for example, necessary media or media supplements, plates or other containers sufficient or adapted to hold such assemblies, a read-out device for the sensor(s), and/or instructions for use of the kit or its components.

Further features and advantages of the compositions and methods described herein include the following. The organized tissue aspect described herein provides a more in vivo-like culture system for screening the activity of biological compounds and offers advantages over disorganized tissue. For example, poorly differentiated cells respond differently to compounds as compared to organized cells in vivo. Also provided are methods for screening a bioactive compound in a tissue which reflects the in vivo cellular organization and gross morphology of the natural in vivo tissue. This organized tissue system offers an efficient and accurate method for screening candidate bioactive compounds for desired biological effects in vitro and in vivo, and permits screening on a long-term, rather than a short-term basis.

Further features and advantages will become more fully apparent in the following description of the embodiments and drawings thereof, and from the claims.

Definitions:

As used herein, by “bioactive compound” is meant a compound which influences the genetic expression profile (e.g., gene up- or down-regulation) biological structure, function, metabolism, or activity of a cell or tissue of a living organism. The candidate bioactive compound will not include the medium or an undefined (i.e., unidentified) component of the medium in which the tissue is tested. The medium may be serum containing or serum-free, as described herein. A component of the medium may be one or more of the following: serum, salt (ions), vitamins, water, selenium, and chicken embryo extract. Preferably, the candidate bioactive compound will consist essentially of the compound to be tested. The candidate bioactive compound is preferably suspended in a basal defined medium. A “bioactive compound” includes, but is not limited to, a small molecule, proteins, including therapeutic proteins, antibodies, antibody fragments, viral and non-viral vectors, RNA, DNA, and fusion proteins. “Bioactive compounds” as referred to herein include, for example, peptides, proteins, fusion proteins, antibodies, antibody fragments, viral and non-viral vectors, RNA and DNA A compound as described herein includes liquids, solids and gases.

As used herein, the term “small molecule” refers to compounds having a molecular mass of less than 3000 Daltons, preferably less than 2000 or 1500, more preferably less than 1000, and most preferably less than 600 Daltons. Preferably but not necessarily, a small molecule is a compound other than an oligopeptide.

“Biological parameter” refers to a measurable characteristic of a biological process of a tissue, cell or organism that is “associated with” a bioactivity and includes but is not limited to measurable chemical changes (e.g. ions, proteins, ATP, receptors, mRNA transcripts, etc.), measurable mechanical changes (e.g. force, size, shape, contractile status) or measurable electrical changes (membrane potential, ion flux, electrical output). For example, the biological parameters of protein degradation, cell damage marker production, and ubiquitination levels are measured to indicate the bioactivity (biological process, for example protein synthesis or creatine kinase release) of muscle wasting. Alternatively, the biological parameters of growth factor production are measured to indicate the biosynthetic and secretory activity of muscle cells. Alternatively, the biological parameters of glucose and lactate production are measured to indicate the metabolic activity of muscle cells.

As used herein, a “tissue” refers to a structure formed in vitro or in vivo from one or more cells. A “tissue” also means an aggregate of cells. In one embodiment, a “tissue” is an aggregate of cells that performs a particular function, for example contraction or relaxation. A “tissue” can comprise cells from a particular anatomic or physiological region. The cells of a “tissue” can comprise a combination of cell types, for example, muscle, fibroblast and nerve cells. A “tissue” of the invention also includes a plurality of cells contained in a location, for example in a well of a tissue culture plate, or at a location of an “array” as described herein, that may normally exist as independent or non-adherent cells in an organism.

A “tissue” as described herein can be disorganized or organized. A “tissue” as described herein can be of any shape, including, but not limited to, for example, a sheet, string, sphere, sling, half-sphere, disc, etc.

“Associated with” refers to an art-accepted scientific correlation between a biological parameter and a biological activity; that is, the biological parameter is what is measured that indicates biological activity.

By “organized tissue” or “organoid” is meant a tissue formed in vitro from a collection of cells having a cellular organization and gross morphology similar to that of the tissue of origin for at least a subset of the cells in the collection. An organized tissue, as used herein, does not include a scaffold which is a pre-formed solid support that imparts or provides short-term (hours to 2 weeks in culture) structure or support to the tissue or is required to form the tissue. An organized tissue or organoid can include a mixture of different cells, for example, muscle (including but not limited to striated muscle, which includes both skeletal and cardiac muscle tissue), fibroblast, and nerve cells, and can exhibit the in vivo cellular organization and gross morphology that is characteristic of a given tissue including at least one of those cells, for example, the organization and morphology of muscle tissue can include parallel arrays of striated muscle tissue. Preferably the organized tissue will include cells that are substantially post-mitotic, and/or aligned substantially parallel to each other and along a given axis of the three-dimensional tissue (with the tissue having x, y and z axes). In an organized tissue with fibers oriented in a lengthwise manner, the length of the tissue is about 0.025 mm-0.250 mm (x, y) and one or more cell layers thick (z). It is preferred that the length of the tissue is in the range of about 0.025 mm-1 mm (x, y) and 0.025 mm to 0.5 mm thick (z). In contrast, a monolayer of cells is typically on the order of 1-10 μm in thickness. An organized tissue can be of any desired width, e.g., about 0.025 mm to about 1 mm or more, and even as much as, for example, 2 mm, 5 mm or 1 cm or more, such that the tissue constitutes a sheet of tissue, for example, as wide or wider than it is long (where for muscle tissue, length is measured parallel to the alignment of the cells). Preferably, an organized tissue will have contraction signaling properties. By “contraction signaling properties” is meant an ability to generate a directed force by changes in overall size, length, and/or shape.

By “in-vivo-like gross and cellular morphology of a tissue of interest” is meant a three-dimensional shape and cellular organization substantially similar to that of the tissue or a component of the tissue in vivo. By “substantially similar to that of the tissue in vivo” is meant that the structure is visibly identical or similar to (for example in terms of morphology or the expression of appropriate marker proteins) or functionally similar to the structure (for example, expresses at least 5% of a marker protein of the native form of the tissue, produces at least 5% of the amount of a protein produced by the structure, or performs an enzymatic reaction at a level that is at least 5% of the level of reaction performed by the tissue).

By “unorganized tissue” or “disorganized tissue” is meant that cells show little in vivo-like intercellular relationship to each other.

As described herein, any “change” in a biological parameter refers to alterations (i.e. an increase or decrease) from a steady state level (for example tension or lack thereof, protein degradation, creatine kinase release, heat shock promoter activity, second messenger activity, growth factor production, glucose and lactate production, and gene up- or down-regulation) of the parameter in a tissue subjected to a candidate bioactive compound. Such a change is indicative of bioactivity. As used herein, a “change” refers to an increase or a decrease of at least 5%, preferably 10-20% and most preferably, 25% or more. A “change” also refers to an increase or a decrease of at least 2-fold, preferably 3-5-fold and most preferably 5-fold or more, for example, 6, 10, 20, 36, 40, 50, 100, 1000-fold or more.

As used herein, an “external stimuli” refers to a stimulus for a muscle tissue (e.g. voltage, force, temperature, chemical, etc.) that does not originate in the muscle tissue and that increases or decreases at least one of the physical, electrical, optical or chemical properties described herein. The increase or decrease in property is measured with a physical, optical, electrical or chemical sensor of the invention.

As used herein, “endogenous” means naturally present, in native, originating from or due to influences from inside of, for example, an organism or a cell.

As used herein, “exogenous” means not naturally present, foreign, originating from or due to influences from outside of, for example, an organism or a cell.

As used herein, “in combination with” means associated with in space, for example, having at least one contact point or located in the same well or tube (with or without at least one contact point), or at the same position of a plate or array (with or without at least one contact point). Thus, in one aspect a tissue that is “in combination with” a sensor is in physical contact with the sensor (e.g., where a sensor directly detects a contractile force), but in another aspect the tissue is not in physical contact with the sensor (e.g., where a sensor measures changes in a property such as birefringence), yet the sensor is present in the culture vessel. A tissue “in combination with” a sensor also includes a sensor that is surrounded by a tissue on one or more (for example 1, 2, 3, 4, 5 or more) sides.

As used herein, the term “container” refers to a structure into which a tissue can be placed ex vivo, such that the tissue is contained within or attached to that structure. A container includes, as non-limiting examples, a culture plate or dish, a well of a multiwell plate or dish, and a sheet of substrate to which a tissue or plurality of tissues as described herein is/are attached. As used herein, “plate” refers to the physical substrate of a culture dish or culture plate, rather than to the combination of a tissue, sensor and culture plate or dish.

As used herein, the term “attached to” means that a tissue is physically adhered to a given surface at at least one point, and preferably at at least two points, such that the tissue is not free in suspension, but rather remains associated with that surface.

As used herein, “viewable microscopically” refers to an object which can be placed on the stage of a dissecting or compound microscope and comprises at least a portion which can be viewed using an ocular of the microscope.

As used herein, “stably associated” refers to an association with a position on a substrate that does not change under washing conditions or under conditions wherein a property of the tissue of the array or sheet is measured.

As used herein, the term “supported by” means that a structure, e.g., a micropost, is physically held in a given position or orientation relative to a surface or a tissue by some substance or structure. Thus, for example, a micropost that is “supported by” an extracellular matrix material will remain, e.g., essentially vertical, or perpendicular to the substrate.

As used herein, the term “in contact with” means physical touching between one entity and another. Thus, a tissue that is in contact with a micropost is physically touching the micropost. The term encompasses both adherent contact (one entity is attached to another) and non-adherent contact (one entity physically touches the other but is not attached).

As used herein, the term “essentially linear” means arranged in approximately a straight line, e.g., an essentially linear path between two points deviates by less than or equal to about 30% of the value of the shortest distance between the two points. A groove that is essentially linear is preferably one in which the path defined by two points on the groove, e.g., points at a distance equal to or greater than the length of a tissue as described herein, deviates from the shortest path between those points by less than about 30%, 20%, 15%, 10%, 5% or less.

As used herein, a “position” refers to a site on a substrate of an array or plate of the invention, that is distinguishable from any other site on the substrate either by eye or by an optical instrument. A “unique position” refers to a position which comprises a single tissue in combination with a sensor.

As used herein, “plate” refers to any of an individual tissue culture plate or a plate comprising multiple wells, for example 6, 12, 24, 48, 60, 72, 96 or 384. A plate can also include, for example, a slide, such as a glass or plastic microscope slide or its equivalent to which a tissue can attach and which can be immersed in culture medium for the maintenance of such tissues. A plate surface can be treated physically or chemically to encourage tissue attachment. A plate of the invention also includes a tube, for example, a microfuge tube that holds for example, 0.75 or 1.50 ml.

As used herein, the term “tub” refers to a depression in a surface into which a suspension of cells can be deposited to form a tissue as described herein. A “tub” can be of any shape, e.g., round, rectangular (including square), triangular, round, etc. In one embodiment, the tubs are elliptical. Non-limiting, preferred dimensions include, for example, a long axis of approximately 25-1000 micrometers, a short axis of approximately 25-1000 micrometers, and a depth or thickness of approximately 25 to 500 micrometers. An elongate (e.g., long axis at least two times as long as the short axis, preferably 2.5 times, 3 times, 3.5 times, 4 times, 4.5 times, 5 times, 6 times, 7 times, 10 times or more) elliptical tub is preferred for promoting a parallel (anisotropic) arrangement of muscle cells deposited into the tub. As used herein, a “tub” is distinct from a “well” in that a “tub” is not necessarily isolated from other tubs on a plate by dividers as would be, for example, one well from another in a multi-well plate. A well can have a plurality of “tubs” in its surface, such that the individual “tubs” in the well are covered by a single volume of medium added to the well. Tubs are preferably arranged in an array in or on a plate as described herein.

As used herein, a “sensor” is a mechanism that detects or measures a change in a tissue as described herein. A “sensor” can detect at least a change in a physical, chemical, optical or electrical property of a tissue of the invention. In one embodiment, a “sensor” measures a change in the length or diameter of a “tissue” of the invention. In another embodiment, a “sensor” measures muscle contraction. In another embodiment, a “sensor” measures muscle relaxation. In another embodiment, a “sensor” measures a change in the temperature of a “tissue” of the invention. In one embodiment, a “sensor” measures a change in the pH of a “tissue” of the invention. A sensor is preferably, but not necessarily of a size that will fit into at least a 384 well plate. The invention also provides for a sensor that can detect or measure a change in a tissue of the invention, wherein the tissue is housed in, for example, a 96, 72, 60, 48, 24, 12 or 6 well plate, in a 35 or 70 mm tissue culture plate or in a 75 ml or 1.5 ml microfuge tube. In one embodiment a sensor of the invention measures a property of a single tissue. In another embodiment, a sensor of the invention simultaneously measures a property of more than one (for example 6, 12, 32, 96, 384) tissues.

As used herein, the term “micropost” refers to one embodiment of a “sensor” as described herein, and comprises a solid member that is attached to or placed in a tissue as described herein. The micropost can be added after formation of the tissue. Preferably the micropost is present in the vessel in which the tissue forms before the formation of that tissue. Preferably a micropost is present in a “tub” comprising a “tissue” as those terms are described herein. A micropost is flexible when placed under tension generated, for example, when tissue surrounding or attached to the micropost contracts.

Flexibility or deflection by a force is calculated by the equation:

$${δ}_{MAX} = \frac{{w}_{o} {L}^{4}}{8 EI}$$

where L is the length of the micropost, E is the elastic modulus, and I is the moment of inertia. [An Introduction to the Mechanics of Solids, Second Edition, S. H. Crandall, N. C. Dahl, and T. J. Lardner, 1978, McGraw-Hill Book Company]. By calculating the moment of intertia of the post, knowing the elastic modulus of the polymer in which the posts were created e.g., using lithography, and knowing the length of the post, one can measure δ and then calculate the load (force). By “flexible” is meant that the micropost has a δ_{MAX} greater than zero. FIG. 14 shows the parameters used in the calculation. The deflection can be measured under a microscope or with a CCD. The posts can waveguide light from the rear or they can be processed such that they have a fluorscent material on the tip.

The posts can range from approximately 5 micrometers to approximately 200 micrometers, most often approximately 5 to approximately 50 micrometers, depending on the length of the post and the elastic modulus of the polymer used in the process. The lengths of the post can range from approximately 10 micrometers to approximately 250 micrometers. If the force from the muscle is small, then longer posts (L) and smaller radii posts are desirable to enhance the deflection. Measurement of the flexion of the micropost provides a measurement of the contraction of a muscle tissue that is attached to or surrounds the micropost. As used herein, “property” includes but is not limited to a physical, chemical, optical or electrical property, for example, the occurrence of muscle contraction, muscle relaxation, the rate (frequency) of muscle contraction or relaxation, the intensity of muscle contraction or relaxation, muscle hypertrophy, muscle atrophy, muscle mass muscle density, muscle vivacity, muscle diameter and muscle length, muscle temperature and muscle pH.

Cell types from which an organized tissue is formed include but are not limited to muscle (smooth and striated), bone, cartilage, tendon, nerve, endothelial and fibroblast.

By “extracellular matrix components” is meant compounds, whether natural or synthetic compounds, which function as substrates for cell attachment and growth.

By “tissue attachment surfaces” is meant surfaces having a texture, charge or coating to which cells may adhere in vitro. Examples of attachment surfaces include, without limitation, stainless steel wire, VELCRO™, suturing material, native tendon, covalently modified plastics (e.g., RGD complex), and silicon rubber tubing having a textured surface. The arrays and plates described herein can comprise a “tissue attachment surface.”

As used herein, the term “external surface,” when referring to a sensor assembly, means a surface in contact with the culture environment. For example, the external surface of a bubble-type sensor is the exterior of the bubble, upon which cells are grown and which is in contact with the culture medium. In contrast, an internal surface of such an assembly is a surface in contact with the hollow space that is in communication with a pressure transducer.

As used herein, the term “elastic material” refers to a material that returns to its original shape after being deformed by application of a force.

By “three-dimensional” is meant an organized tissue having x, y and z axes wherein x and y of the axes are at least 0.025 mm with z at least 0.025 mm thick, and wherein 1, 2 or all of the axes are as great as 20 cm. Preferably, a three-dimensional tissue is capable of contraction signaling. By “contraction signaling” is meant the ability to generate a directed force by changes in overall size, length, and shape. Preferably a three-dimensional muscle tissue is comprised of cells that have fused in art organized manner similar to the tissue of origin; for example the organization and morphology of muscle tissue may include parallel arrays of striated muscle tissue.

By “at least a subset of cells” is meant at least two cells, preferably at least 10% of the cells of the tissue, and more preferably at least 25% of the cells.

As used herein, a “plurality of cells” refers to more than one cell, e.g., 2, 3, 4, 5, 10, 20, 50, 100, 1000, 10,000 or more cells.

By “substantially post-mitotic cells” is meant a tissue, organoid or population of cells in which at least 50% of the cells are non-proliferative. Preferably, tissues including substantially post-mitotic cells are those in which at least 80% of the cells are non-proliferative. More preferably, tissues including substantially post-mitotic cells are those in which at least 90% of the cells are non-proliferative. Most preferably, tissues including substantially post-mitotic cells are those in which 99% of the cells are non-proliferative. Cells of a tissue retaining proliferative capacity can include cells of any of the types included in the tissue. For example, in striated muscle tissues such as skeletal muscle tissues, the proliferative cells can include muscle stem cells (i.e., satellite cells) and fibroblasts.

By “aligned substantially parallel” is meant that cells are aligned substantially parallel to each other and along a given axis of the three-dimensional tissue, which is preferably the longest axis of the tissue (with the tissue having x, y and z axes).

By “substantially all of the cells” is meant at least 90% and preferably 95-99% of the cells.

By “monolayer” is meant a single cell layer.

By “differentiated” is meant cells with numerous mature-like characteristics, either chemical or physical.

By “terminally differentiated” is meant that a cell or tissue is not capable of further proliferation or differentiation into another cell or tissue.

As used herein, an “array” means a plurality of tissues in combination with a sensor, stably associated with a substrate. The term array is used interchangeably with the term “microarray”, however, the term “microarray” is used to define an array which has the additional property of being viewable microscopically. An array preferably has at least two tissue moieties, and preferably more, e.g., at least 3, at least 4, at least 5, at least 10, at least 20, at least 24, at least 48 or more, e.g., at least 96 or more, e.g., at least 100, 200, 300 or e.g., 384 or more.

By “of a type that is not normally present in the cells” is meant foreign to the cell.

By “in an amount that is not normally produced by the cells” is meant at least 5% above or below the amount normally produced by the cells or tissue, preferably at least 10% above or below, more preferably 50-100% above or below, or greater than 100% above the amount normally produced by the cells or tissue, or, for example, at least 2 fold, 5 fold, 10 fold, 20-fold or more above the amount normally produced by the cells or tissue.

By “heterologous gene” is meant a DNA sequence that is introduced into a cell.

By “foreign DNA sequence” is meant a DNA sequence which differs from that of the wild type genomic DNA of the organism and may be extra-chromosomal, integrated into the chromosome, or the result of a mutation in the genomic DNA sequence.

By “muscle wasting” is meant a loss of muscle mass due to reduced protein synthesis and/or accelerated breakdown of muscle proteins, including for example, as a result of activation of the non-lysosomal ATP-ubiquitin-dependent pathway of protein degradation.

By “attenuation of muscle wasting” is meant preventing or inhibiting muscle wasting.

By “short-term” is meant a length of time in which cells are viable for a period that does not exceed but includes 14 days.

By “long-term” is meant a length of time in which cells are viable that is more than 14 days and as long as 30 days, 60 days and 90 days or more.

“Contacting” refers to exposing a tissue or cells thereof, to a compound, or mixing the tissue and the compound.

As used herein in reference to monitoring, measurements or observations in assays described herein, the term “real-time” refers to that which is performed contemporaneously with the monitored, measured or observed events and which yields as a result of the monitoring, measurement or observation to one who performs it simultaneously, or effectively so, with the occurrence of a monitored, measured or observed event. Thus, a “real time” assay or measurement contains not only the measured and quantitated result, such as muscle contraction, but expresses this in real time, that is, in hours, minutes, seconds, milliseconds, nanoseconds, picoseconds, etc. Shorter times exceed the instrumentation capability; further, resolution is also limited by the folding and binding kinetics of polypeptides.

### BRIEF DESCRIPTION OF THE DRAWINGS

FIG. 1 shows a diagram of one embodiment of a micropost array.

FIG. 2 shows the various dimensions associated with members of one embodiment of a micropost array.

FIG. 3 shows a schematic illustration of the use of an automatic dispenser to deposit tissue precursors into wells or tubs in an array.

FIG. 4 shows a schematic illustration of the use of grooves in a substrate to assist the preparation of tissues.

FIG. 5 diagrams the measurement of contractile force using microposts.

FIG. 6 diagrams two of the ways changes in the distance between microposts can be measured.

FIG. 7 shows a patterned micro-post array.

FIG. 8 shows a patterned micropost array arranged in a series of wells.

FIG. 9 shows various possibilities for the dimensions of microposts.

FIG. 10 shows various possibilities for different lattice unit cells for micropost arrays.

FIG. 11 shows one possibility for sub-patterning of microposts within a single well.

FIG. 12 shows schematic diagrams of two possible sensor-tissue arrangements. 12a shows a “drum head” arrangement; 12b shows a “sphere” or “bubble” arrangement; and 12c shows a photograph of a bubble-type sensor.

FIG. 13 shows a schematic of an array of “bubble” sensor devices.

FIG. 14 shows a schematic of a micropost and the parameters for determining the deflection of the micropost under tension.

FIGS. 15A and B shows two views of an embodiment in which a muscle tissue is prepared in an anisotropic tub comprising microposts and flanked by electrodes that permit the application of an electrical field.

### DETAILED DESCRIPTION

Compositions and methods described herein provide tissue in an in vitro or ex vivo context, in combination with a sensor that permits the measurement of a response of the tissue to a stimulus or environmental change. Such compositions and methods permit, for example, the screening of tissue for the effects of agents or treatments that elicit a desired response or otherwise have a desired effect on the tissue. Thus, using the compositions and methods described herein, one can screen a candidate biologically active (“bioactive”) compound for its biological effects on a tissue or cells of a tissue. The methods and compositions are suited, for example, for screening for effects on organized or disorganized tissue or cells of such tissues. The methods and compositions are particularly well suited for screening for effects on organized tissue or cells of an organized tissue. The methods permit the use of human tissues that possess or retain at least some biochemical and mechanical functions of similar tissues in vivo, and are long-lived, e.g., on the order of weeks (e.g., one week, two weeks, three weeks, four weeks, five weeks, etc.), months (e.g., one month, two months, three months, four months, five months, etc.) or more.

Preparation of a Tissue:

The preparation of tissue is known in the art and will vary depending upon the tissue type one wishes to study. The tissue is preferably prepared in a manner that preserves one or more differentiated properties of the corresponding tissue in vivo. The tissue can be derived from human or non-human animal sources. In one aspect, the tissue can be derived, for example, from a human that is healthy or, alternatively, from a human that suffers from a disease of interest (e.g., one affecting that tissue). Tissue derived from healthy or diseased human individuals can permit prediction of the activity of a drug or drugs in humans.

For embodiments where a tissue is contained in a plate or a well of a multi-well plate, the tissue is of a shape and size that can be contained in the plate or well.

Tissues applicable to the methods and compositions described herein encompass any tissue that can be formed in vitro by methods known to those of skill in the art. A tissue can be produced, for example, as described in U.S. Pat. Nos. 4,940,853 and 5,153,136, the contents of which are incorporated by reference herein. A tissue of the invention can also be prepared as described in U.S. Pat. No. 5,869,041. A preferred tissue is a muscle tissue.

In some embodiments, the tissue can include primary human tissue, primary non-human animal tissue, and primary tissue obtained from donors with specific disease states (e.g., where the tissue is muscle tissue, the disease state can include, atrophy, cardiac disease, etc.). In these instances, the disease states can be associated with existing conditions or, alternatively, can be induced through artificial means, e.g., genetic manipulation, such as occurs in knock-out animals or in transgenic animals, including, for example, knock-in animals.

The use of genetic manipulation techniques can permit the identification of pathways affected by a given drug or stimulus. For example, when tissue comprising a knock-out lacks responsiveness to a drug or stimulus, the pathway affected by the drug or stimulus is highlighted by the knock-out. Similarly, where tissues are prepared using cells from an individual with a disease or disorder affecting that tissue, a response or lack of a response to a drug or stimulus (e.g., a drug with known effects against normal or abnormal tissues) can be indicative of the nature of the disease. This approach can also be used to rapidly screen tissue derived from an individual to predict the efficacy of one of a panel of drugs on that individual's disease symptoms. Alternatively, a panel of tissues, each with a knock-out or knock-in affecting a known pathway can be used to rapidly screen the effect of a given drug candidate on that pathway, both initially, and as a function of extended continuous or repeat dosages.

In one aspect, the tissue is muscle tissue, including, for example, smooth muscle, striated muscle and cardiac muscle. Muscle tissue can be prepared, for example, as described below.

A tissue as described herein is of a size and shape whereby it can survive initially, in vitro and in vivo, via a diffusion of nutrients into the organized tissue, and is also three-dimensional. For embodiments wherein the tissue is housed in a well of a plate, or a tissue culture dish, the tissue of the invention is of a size and shape that will fit into a tissue culture well or dish. The well can be a standard 384, 96, 72, 60, 32, 16, 12, or 6-well plate, a standard tissue culture plate or dish with a diameter of 35 mm, 70 mm or more, or a standard 75 ml or 1.5 ml microcentrifuge tube. Also possible are custom-sized and -shaped wells and plates of any dimensions, as well as tissues that are prepared to fit into the custom sized and shaped wells and plates.

A tissue as described herein can have at least one contact point, and possibly more than one, for example, 2, 5, 10, 50, 100, 1000 or more, with the sensor.

The tissue can be prepared in the presence or absence of a sensor. That is, the tissue can be prepared in a container such that the sensor is integrated into or attached to the tissue, e.g., as when the tissue grows or is deposited on, around or in contact with the sensor. Alternatively, the tissue can be prepared independent of the sensor, with the sensor later being placed in communication with the tissue.

As used herein, the term “independent from the sensor” means that tissue exists separately from the sensor, such that if the sensor is removed, the tissue will maintain substantially the same morphology and arrangement. A tissue that is first prepared and then placed in communication with the sensor is “independent from the sensor.”

As the term is used herein, the term “not independent from the sensor” means that the tissue and sensor are associated in a manner such that removal of the sensor would substantially alter the morphology and/or arrangement of the tissue. Where the tissue is grown or deposited on a surface of the sensor itself, the tissue is “not independent from the sensor.”

In certain embodiments, a “tissue” as described herein is under tension. As used herein, “tension” means stress resulting from cell organization and/or fusion or reorganization, for example resulting from the fusion of myoblasts into myofibers, elongation, stress resulting from stretching, for example from one or more external tissue attachment points or surfaces, or internally derived tension, for example, resulting from internal pressure, for example, as would be exerted by a coalescing of cells on each other due to their confinement to a particular internal area, for example, a well of a tissue culture plate.

Organized tissues having in vivo-like gross and cellular morphology can be produced in vitro from the individual cells of a tissue of interest. As a first step in this process, disaggregated or partially disaggregated cells can be mixed with a solution of extracellular matrix components to create a suspension. This suspension can then be placed in a vessel having a three dimensional geometry which approximates the in vivo gross morphology of the tissue and includes tissue attachment surfaces coupled to the vessel. The cells and extracellular matrix components are then allowed to coalesce or gel within the vessel, and the vessel is placed within a culture chamber and surrounded with media under conditions in which the cells are allowed to form an organized tissue connected to the attachment surfaces.

By “extracellular matrix components” is meant compounds, whether natural or synthetic compounds, which function as substrates for cell attachment and growth. Examples of extracellular matrix components include, without limitation, collagen, laminin, fibronectin, vitronectin, elastin, glycosaminoglycans, proteoglycans, and combinations of some or all of these components (e.g., Matrigel™, Collaborative Research, Catalog No. 40234).

By “tissue attachment surfaces” is meant surfaces having a texture, charge or coating to which cells may adhere in vitro. Examples of attachment surfaces include, without limitation, stainless steel wire, VELCRO™, suturing material, native tendon, covalently modified plastics (e.g., RGD complex), and silicon rubber tubing having a textured surface. Attachment surfaces can also include, for example, the surface of microposts as described herein. The arrays and plates described herein can comprise a “tissue attachment surface.”

Although this method is compatible with the in vitro production of a wide variety of tissues, it is particularly suitable for tissues in which at least a subset of the individual cells are exposed to and impacted by mechanical forces during tissue development, remodeling or normal physiologic function. Examples of such tissues include muscle, bone, skin, nerve, tendon, cartilage, connective tissue, endothelial tissue, epithelial tissue, and lung. More specific examples include skeletal and cardiac (i.e., striated), and smooth muscle, stratified or lamellar bone, and hyaline cartilage. Where the tissue includes a plurality of cell types, the different types of cells can be obtained from the same or different organisms, the same or different donors, and the same or different tissues. Moreover, the cells can be primary cells or immortalized cells. Furthermore, all or some of the cells of the tissue can contain a foreign DNA sequence (for example a foreign DNA sequence encoding a receptor) which indicates a response to a bioactive compound or otherwise modifies the tissue to facilitate an assay.

The composition of the solution of extracellular matrix components will vary according to the tissue produced. Representative extracellular matrix components include, but are not limited to, collagen, laminin, fibronectin, vitronectin, elastin, glycosaminoglycans, proteoglycans, and combinations of some or all of these components (e.g., Matrigel™, Collaborative Research, Catalog No. 40234). In tissues containing cell types which are responsive to mechanical forces, the solution of extracellular matrix components preferably gels or coalesces, such that the cells are exposed to forces associated with the internal tension in the gel.

An apparatus for producing a tissue in vitro having an in vivo-like gross and cellular morphology includes a vessel having a three dimensional geometry which approximates the in vivo gross morphology of the tissue. The apparatus also includes tissue attachment surfaces coupled to the vessel. Such a vessel can be constructed from a variety of materials which are compatible with the culturing of cells and tissues (e.g., capable of being sterilized and compatible with a particular solution of extracellular matrix components) and which are formable into three dimensional shapes approximating the in vivo gross morphology of a tissue of interest. In one aspect, the tissue attachment surfaces (e.g., stainless steel mesh, VELCRO™, or the like) are coupled to the vessel and positioned such that as the tissue forms in vitro the cells can adhere to and align between the attachment surfaces. Tissue attachment surfaces can be constructed from a variety of materials which are compatible with the culturing of cells and tissues (e.g., capable of being sterilized, or having an appropriate surface charge, texture, or coating for cell adherence).

Where necessary, tissue attachment surfaces can be coupled in a variety of ways to an interior or exterior surface of the vessel. Alternatively, the tissue attachment surfaces can be coupled to the culture chamber such that they are positioned adjacent to the vessel and accessible by the cells during tissue formation. In addition to serving as points of adherence, in certain tissue types (e.g., muscle), the attachment surfaces allow for the development of tension by the tissue between opposing attachment surfaces.

In one aspect, a vessel for producing an organized tissue that is suitable for the in vitro production of a skeletal muscle organoid preferably has a substantially semi-cylindrical shape and tissue attachment surfaces coupled to an interior surface of the vessel (Shansky et al., In Vitro Cell Develop. Biol. 33: 659-661, 1997). The vessel can be, for example part of a plate as described herein, wherein the plate has depressions or grooves (also referred to as “tubs”) into which cells can be deposited. The shape of the tubs will facilitate the organization of such cells into a tissue. Non-limiting examples of tub shapes and dimensions are described herein below in the section titled “Micro Post Arrays.”

Using an apparatus and method as generally described above, a skeletal muscle organoid having an in vivo-like gross and cellular morphology is produced in vitro. During skeletal muscle development embryonic myoblasts proliferate, differentiate, and then fuse to form multi-nucleated myofibers. Although the myofibers are non-proliferative, a population of muscle stem cells (i.e., satellite cells), derived from the embryonic myoblast precursor cells, retain their proliferative capacity and serve as a source of myoblasts for muscle regeneration in the adult organism. Therefore, either embryonic myoblasts or adult skeletal muscle stem cells may serve as one of the types of precursor cells for in vitro production of a skeletal muscle organoid.

In another aspect, tissue is prepared on a surface, e.g., a plate, having tubs into which muscle cells are deposited and which promote the formation of small units of unidirectionally arranged muscle tissue. Exemplary dimensions of the tubs are described below in the context of micropost arrays, but are applicable to any arrangement of tubs. In one embodiment, the tubs can contain microposts as described herein, such that the tissue forms and can become organized in contact with and between the microposts. In another embodiment, the microposts are contacted with the tissue after it has been formed in the tubs, as where, for example, a probe apparatus comprising a set of microposts is lowered into contact with the tissue after it is formed in the tubs. In one aspect, an advantage of tissue/sensor arrangements described herein is that their long-lived nature can permit the monitoring of the effect(s) of a drug or drug combination over time (e.g., days, weeks, months) and over a number of doses (e.g., two, three, four, 10, 20, 50, etc.) to determine not just the effect(s) of the drug(s), but also any changes in such effect(s) occurring over time and with repeated dosing.

The use of individual tissues in separate wells, e.g., separate wells of a multiwell plate, or in separate tubs in one or more wells permits the rapid measurement of bioactivity in multiple tissues. In one embodiment, the tissues in different wells or tubs can be the same, e.g., prepared from cells of the same tissue of the same individual or from the same tissue of individuals of the same species. Alternatively, the tissues can be prepared from different cells of the same or different individuals, thereby permitting the contemporaneous monitoring of bioactivity against different tissues, e.g., cardiac vs. striated muscle, or even for example, muscle vs. another tissue type, e.g., liver or another tissue type.

An array of wells comprising tissue can be subjected to different drug treatments and monitored in a high throughput fashion. The ability to do so in small volumes provides another advantage, for example, as it reduces the amounts of test compound and reagents needed, among others. Similarly, when the tub format is used (see below), multiple tissues in individual tubs can be monitored closely in time. As with the tissues in different wells, tissues in different tubs can have different sources or compositions. This can be achieved, for example, by loading the individual tubs with tissue precursors from different sources, e.g., from different individuals or from different types of tissue from the same or different individuals.

Sensors:

A sensor as described herein permits the measurement of a parameter associated with a tissue as such parameters are described herein. In one embodiment the “sensor” is a physical sensor, e.g., an oscilloscope (for example Agilent 500 MHz) or a pressure transducer (for example Omega PX655). Among the parameters such a sensor can measure is muscle contraction.

In another embodiment, the “sensor” is an optical sensor. In one aspect, an optical probe includes but is not limited to a laser, a polarizer, an optical detector and an oscilloscope or multimeter. An optical probe of the invention measures, e.g., the contraction of a muscle, by detecting changes in the birefringence of the muscle. Since a muscle can be highly organized, it possesses the property of birefringence. (Birefringence is the term used to describe a material that possesses two different indices of refraction, which depends on the polarization of the incident light.) As the muscle contracts, an optical probe can detect small changes in birefringence by measurement of the intensity of polarized light incident on the sample in reflection or transmission.

In another embodiment, the sensor is a chemical sensor that measures pH or changes in the pH of a tissue. In another embodiment, a sensor measures changes in the temperature of a tissue (e.g., where the sensor comprises a thermometer).

There are no limitations to the shape of a sensor useful according to the invention. A sensor as described herein can be of any shape, e.g., a sheet, a string, a sphere, a sling, a drum, a half-sphere, a disc, a dumbbell, a roll or a drum head. In one embodiment, a sensor comprises a hollow cavity, for example, into which a compound can be placed or which comprises air or another gas, or a liquid.

In one embodiment, a “sensor” is used to detect or measure a change in a single tissue. In another embodiment, a sensor is used to simultaneously measure a property in multiple tissues. In certain embodiments, a sensor is used to measure a property in a first tissue, is removed from the first tissue and is either reintroduced into the first tissue or is introduced into a second tissue to provide an additional or second measurement.

In one aspect, a sensor is not in contact with a tissue.

In another aspect, a sensor has at least one contact point with a tissue as described herein. In one embodiment, a sensor has more than one contact point with a tissue, for example, 2, 5, 10, 50, 100, 1000, or more. In one embodiment, a sensor can be introduced into a tissue of the invention after the tissue has formed a three-dimensional structure. In another embodiment, the tissue is formed in the presence of a sensor.

A sensor as described herein can be elastic or solid. A sensor as described can be of a range of porosity or permeability such that diffusion of a compound of interest, across a sensor, can be measured. The pore size of a material comprised by a sensor of the invention can be from 1 nm to 100 micrometers or more, for example 1 nm, 10 nm, 20 nm, 30 nm, 40 nm, 50 nm, 60 nm, 70 nm, 80 nm, 90 nm, 100 nm, 1 μm, 10 μm, 20 μm, 30 μm, 40 μm, 50 μm, 60 μm, 70 μm, 80 μm, 90 μm, 100 μm or more. The porosity or permeability of a sensor of the invention is selected based on the diffusion properties of the compound of interest and the application for which the sensor is to be used. The methods described herein provide for a “sensor” that is made of or comprises material that is either exogenous to the tissue or endogenous to the tissue (for example, extracellular matrix material). The invention also provides for a “sensor” that is made of or comprises a combination of materials that are endogenous and exogenous to the tissue.

A “physical sensor” as described herein measures a physical property, including but not limited to the occurrence of muscle contraction, muscle relaxation, the rate (frequency) of muscle contraction or relaxation, the intensity of muscle contraction or relaxation, muscle hypertrophy, muscle atrophy, muscle mass muscle density, muscle vivacity, muscle diameter and muscle length, and muscle temperature. A physical sensor can detect a response to an exogenous stimulus (for example an exogenous compound, e.g., a drug, or a physical stimulation). A physical sensor can also respond to an endogenous stimulus, for example endogenously initiated or stimulated contraction of a tissue. A “physical” sensor detects a differential pressure for example, recorded by a differential pressure transducer and read out by any one of an ammeter, voltmeter, multimeter or oscilloscope.

Temperature can be measured using a “physical sensor” that is a thermometer or temperature probe.

In one aspect, an organized tissue produced as described herein can be tethered to attachment points at either end of a culture vehicle. One or both ends of the tissue attachment sites is/are connected to a force transducer instrument (e.g. Model 400A Series Force Transducer Systems, Aurora Scientific, Inc.) that is connected to an oscilloscope to be used for monitoring the readout. In another embodiment the organized tissue is grown around the force transducer instrument. In another embodiment the organized tissue is impaled by the force transducer instrument.

The addition of certain agents to the media or perfusate of the tissue results in a change in the dimensions, contractile state, contractile frequency or force generated of or by the tissue. This change is detected by the attached force transducer and read out on the oscilloscope or a comparable apparatus.

This system can detect a range of frequencies from 0.5 Hz to 100 kHz, a change in dimensions in the range of approximately 0.1 μm to 1 cm and a change in force in the range of approximately 0.001 μg to 10,000 g.

An apparatus capable of mechanically stimulating the tissue with a known force (0.001 μg to 10,000 g), distance (0.1 μm to 1 cm) or frequency range (0.01 Hz to 100 kHz) can also be included in this system and used for measurement, calibration, etc. purposes. An example of this type of apparatus is the Series 300B Lever Systems (Aurora Scientific, Inc., Ontario, Canada).

An “optical sensor” as described herein measures an optical property including but not limited to birefringence, scattering, reflection or transmission. The occurrence of muscle contraction, the rate/frequency of muscle contraction, the intensity of muscle contraction, muscle hypertrophy, muscle mass and muscle length are detectable events that can be measured with an optical probe. These events manifest themselves in certain optical properties that are measurable. For example, muscle contraction is expected to result in subtle changes in the birefringence of the muscle, which can be detected in transmission or reflection of polarized light off the sample. Another example includes changes in muscle length, which change the birefringence and are therefore detectable. Another example includes the monitoring of subtle differences in light scattering as the muscle is contracting. The frequency of these events can also be measured by monitoring the transmission, reflection or scattering data on an oscilloscope to probe the event in the time domain. An “optical sensor” measures an optical property by sending an optical signal into a detector (for example a charge coupled device (CCD) or a photodiode) that is read by any one of an ammeter, voltmeter, multimeter or oscilloscope.

In one aspect, a tissue produced as described herein, from cells transfected with a vector expressing an autofluorescent marker, for example the Green Fluorescent Protein (GFP), is connected to a light source in an instrument capable of measuring fluorescence. If a secreted form of the fluorescent maker is used, constant real-time marker production can be measured directly in the culture medium. If the marker is expressed intracellularly, the incident light beam is aimed directly at the organized tissue. The amount of fluorescent marker is quantitated by fluorescence using a multiwell plate fluorescence unit in which the tissues are grown.

Alternatively, a tissue can be produced from cells stably transfected with a vector expressing secreted alkaline phosphatase (SEAP). The amount of secreted SEAP is measured by fluorescence or chemiluminescence in an aliquot of the culture medium following the addition of the chemiluminescent substrates CSPD or MUP. Alternatively, if the presence of the substrates is not detrimental to the cultured tissues, these substrates are added directly into the culture medium contained in the culture wells, and the amount of secreted SEAP measured by fluorescence or chemiluminescence.

As described herein, an “electrical sensor” measures an electrical property including but not limited to resistance, capacitance or current. An “electrical sensor” measures an electrical property by sending an electrical signal into an amplifier or reading the electrical signal directly using any one of an ammeter, voltmeter, multimeter or oscilloscope.

The occurrence of muscle contraction, the rate/frequency of muscle contraction, the intensity of muscle contraction, muscle hypertrophy, muscle mass and muscle length can be subjected to electrical measurements. Since an electrical signal can be sent through the tissue sample, the response of the muscle or an electrical property of the tissue can be measured. For example, the capacitance of a tissue sample can be monitored when placed between two electrodes. For an aligned contracting muscle, the capacitance is expected to change due to subtle changes in muscle length, which manifests itself in the dielectric properties of the tissue. The dielectric constant is related to the capacitance. Another example includes a ‘carpet’ of conducting pillars (not necessary to be on the nanoscale) which comes in contact with the muscle. As the muscle contracts, the conductivity (resistivity) of the pillars can change and can be monitored with an oscilloscope. The change is a result of the pillars changing their proximity to each other, thereby resulting in subtle changes in the conductivity of pillars measured at the edges of the samples.

In one aspect, a tissue produced as described herein is tethered to attachment points at either end of a culture vehicle (open system, closed cartridge module, etc.). One or both ends of the tissue attachment sites are connected to an electrical/ionic output measuring instrument that is connected to an oscilloscope to be used for monitoring the readout. In another embodiment, organized tissue is grown around the electrical/ionic output measuring instrument. In another embodiment, organized tissue is impaled by the electrical/ionic output measuring instrument.

The addition of certain agents to the media or perfusate of the tissue will result in a change in the electrical output of the tissue. This change will be detected by either attached surface EMG electrodes or an attached force transducer and read out on the oscilloscope or a comparable apparatus. The range of electrical output detected is from 1 μV to 1000 μV. An apparatus capable of mechanically stimulating organized tissue with a known force (0.001 μg to 10,000 g), distance (0.1 μm to 1 cm) or frequency range (0.01 Hz to 100 kHz) can also be included in this system and used for measurement, calibration, etc. purposes. An example of this type of apparatus is the Series 300B Lever Systems (Aurora Scientific, Inc., Ontario, Canada).

As described herein, a “chemical sensor” measures a chemical property including but not limited to pH, salt or other ion concentration and oxidation or reduction status. pH can be measured using a “physical sensor” that is a pH meter. Salt or ion concentrations are often measured by changes in conductance or resistance. (It is noted that pH is also frequently measured as a difference in electrical potential; however, as used herein, pH is considered a chemical property.) A “chemical sensor” can also be used to determine the presence, absence or a change in the level of a gene, nucleic acid or gene product of interest. To the extent that a fluorescent reporter protein is employed to measure gene expression, a biochemical property, a fluorescence or other optical detector used to detect the presence of reporter gene product can also be considered a “chemical” sensor. In one embodiment, the chemical sensor comprises, e.g., a PCR machine used to monitor an RT-PCR reaction. In this instance, the PCR machine provides an indirect read out of bioactivity, in that an intermediate nucleic acid amplification step is required to generate a signal. In another embodiment, where, for example, a direct read-out is preferred, the sensor does not comprise a PCR machine.

A chemical sensor can also detect the presence of a protein, e.g., on the basis of binding of a target protein, e.g., one expressed by a tissue as described herein. In one embodiment, for example, the chemical sensor measures surface plasmon resonance changes induced by the binding of a target protein to a protein or other binding partner immobilized on a chip. The measurement of, e.g., protein or other biochemical binding, by changes in surface plasmon resonance is well known in the art.

In one embodiment a sensor is used to measure a property in a single tissue.

In another embodiment, a sensor can be used to simultaneously measure a property in multiple tissues. Also contemplated is an apparatus that comprises multiple sensors that are connected to each other or to a common read-out device and can be used to simultaneously measure a property in multiple tissues.

In certain embodiments, a sensor is used to measure a property in a first tissue, is removed from the first tissue and is either reintroduced into the first tissue or is introduced into a second tissue to provide an additional or second measurement. In one embodiment, a sensor can be used for multiple (i.e., more than one, 2, 3, 4, 5, 10, 20, 30, 40, 50, 100 or more) measurements, and with more than one (i.e., at least 2, 3, 4, 5, 10, 32, 96, 384 or more) tissue. In one embodiment, an individual sensor can be used to make multiple (i.e., more than one, for example, 2, 3, 4, 5, 10, 20, 30, 40, 50, 100 or more) measurements, and with more than one tissue (i.e., for example, 2, 3, 4, 5, 10, 20, 30, 40, 50, 100 or more). In another embodiment, multiple sensors or arrays of sensors (i.e., more than one, for example, 2, 3, 4, 5, 10, 20, 30, 40, 50, 100 or more) can be used for multiple measurements (i.e., more than one, for example, 2, 3, 4, 5, 10, 20, 30, 40, 50, 100 or more) and with more than one tissue (i.e., more than one, for example, 2, 3, 4, 5, 10, 20, 30, 40, 50, 100 or more).

A sensor that is “in combination” with a tissue includes a sensor that is independent from a tissue, and, in certain embodiments, can be removed from a tissue and reintroduced into the same tissue or introduced into a second tissue. (“Introduced into” is intended to encompass not only the situation in which a sensor is physically inserted into a tissue, as in, e.g., the way in which a needle is inserted into a tissue, but also the situation in which the sensor is merely placed in contact with or in close proximity with the tissue, such that a parameter can be measured.) A sensor that is “in combination” with a tissue also includes a sensor that is not independent from and cannot be removed from a tissue and then reintroduced into the same tissue or introduced into another tissue.

In one embodiment, a tissue can be formed independently from a “sensor”. A “sensor” can be brought in contact with the cells of a tissue after or prior to tissue formation. In another embodiment, the tissue is formed in the presence of a sensor that can be removed from the tissue. In another embodiment, a “sensor” can be used to measure or detect a property of a tissue, and then removed from the tissue following the measurement or detection step. Such a sensor can be removed from a first tissue and then reintroduced into the first tissue or introduced into a second tissue, and used to measure or detect a property of, the first tissue or at least one additional tissue. In one embodiment, the sensor is used multiple times, for example to measure a property in a tissue in each of 384 wells of a 384 well plate. In another embodiment, a sensor comprises multiple sensors, for example, a sensor includes a sensor that can be used to simultaneously measure a property in each of the tissues in a 384 well plate.

A “sensor” that is “not independent from” a tissue has at least one point of contact with a tissue. A “sensor” that is “not independent from” a tissue cannot be removed from a tissue and then reintroduced into the same tissue or be introduced into a second tissue.

Muscle contraction, muscle relaxation and muscle length can be measured by using a physical sensor, for example, an oscilloscope (for example Agilent 500 MHz) and a pressure transducer (e.g. Omega PX6555). Muscle contraction rates/frequency are measured by increases or decreases in pressure detected by the pressure transducer. In another embodiment, muscle contraction or muscle relaxation is measured by detecting changes in the birefringence of the muscle, using an optical sensor, for example an optical probe (e.g. laser), a polarizer, an optical detector, an oscilloscope or a multimeter.

“Muscle hypertrophy” or muscle “atrophy” can be measured by using a physical sensor, for example an oscilloscope and a pressure transducer to measure the change in pressure from a first point in time to a second point in time. Pressure measurements can be taken periodically over a defined time interval to measure the progression of muscle hypertrophy or atrophy over time.

As used herein, “device” refers to a device that is used in combination with a sensor of the invention to provide a readout for a change in a physical property of a tissue of the invention.

Any of the devices are used with a sensor to measure changes in any of muscle contraction, muscle length, muscle mass or muscle density, in response to external or internal stimuli.

In one embodiment, a device that is used in combination with a physical sensor measures the amperes (or volts) that are produced by a differential pressure transducer. For example, the output of a pressure transducer (for example Omega PX6555) is read by an ammeter (for example provided by Omega). Alternatively, the output of a pressure transducer is measured by any of an oscilloscope, ammeter, voltmeter, or multimeter. The data can be acquired by a computer using for example an HPIB interface card (HP version) or a GPIB interface card (industry standard). In one embodiment, the HPIB card is used in combination with the HP-VEE software (Hewlett Packard). In another embodiment the GPIB card is used with Lab View Software (National Instruments).

In one embodiment, a device that is used in combination with an optical sensor is an oscilloscope, an ammeter; a voltmeter or a multimeter. In that instance, as above, the data can be acquired by a computer using a HPIB interface card (HP version) or GPIB interface card. In one embodiment, also as above, the HPIB card is used with HP-VEE software (Hewlett Packard). In another embodiment the GPIB card is used in combination with Lab View Software (National Instrument).

In one embodiment, a device that is used in combination with an electrical sensor is an oscilloscope, an ammeter, a voltmeter or a multimeter. The data can be acquired by a computer as above.

In one embodiment, a “device” that is used in combination with a chemical sensor comprises, for example, a fluorimeter, a spectrophotometer, a luminometer or a phosphorimager. Chemical assays are used to detect the presence, absence or change in a level of a chemical, protein or gene product (e.g., a transcript). Chemicals that can be used to provide a read-out of a change in a property of a tissue, e.g. a muscle tissue, include, for example: chlorzoxazone, a skeletal muscle relaxant, used to treat local muscle spasms; acetylcholine, which relates to cardiovascular, smooth, and skeletal muscle (physiological) contraction; methylcellulose, a bulk laxative active on smooth muscle; morphine, which has few cardiac effects, but also influences smooth muscle contraction, GI muscle spasms, constipation, and causes skeletal muscle rigidity; Tolterodine, a bladder antispasmodic that mediates urinary bladder contraction; dopamine, which functions in large doses as a cardiac stimulant; and esmolol, an antiarrhythmic. Further, enzymes or the activity of enzymes, such as creatine phosphokinase, lactic dehydrogenase, myoglobin and troponins T and I can be assayed as a chemical read-out of muscle status, particularly as a read-out of cardiac muscle status. Additional chemicals that can be assayed as a measure of a change in a tissue parameter include, for example, nucleic acids and polypeptides. Nucleic acids can be detected, for example, using a thermal cycler. Polypeptides can be detected, for example, using immunoassay technology or specific binding partners to the polypeptides of interest.

A substrate of an array as described herein can be made from any of silicon, rubber, polymer, elastomer, plastic, glass or any other material that is compatible to the attached tissue and assists the growth of tissue. Generally, to be compatible to the attachment of tissue, a material should be hydrophilic, as the “wettability” of the surface is critical to cell and tissue attachment. For plastic surfaces, e.g., polystyrene, the oxygen content of the surface directly influences the wettability and thus, the compatibility for tissue attachment. The surface roughness of a substrate also influences the attachment of tissues, with rougher surfaces generally providing better attachment than smoother surfaces. As an example, U.S. Pat. No. 6,617,152 and references cited therein describe surface treatments useful for increasing the cell attachment characteristics of a surface.

The thickness of the substrate of an array is 1 μm to 150 μm or more, for example, 200 μm, 300 μm, 400 μm, 500 μm, 1 mm, 10 mm, 100 mm or more.

In one embodiment, a tissue is attached to an “array” independently of a sensor. In another embodiment, a tissue in combination with a sensor is attached to an “array”.

Screening Methods:

A method of screening a candidate compound for bioactivity in a tissue includes culturing a tissue in the presence or absence of a candidate bioactive compound, and, using a sensor, measuring a biological parameter of the tissue or one or more cells of the tissue. In one embodiment, a measurement of a biological parameter can be made via a sensor that is in contact with the tissue.

A candidate bioactive compound can be screened, for example, in an organized tissue comprising, for example, muscle cells. A biological parameter measurable in muscle tissue, and of interest in the invention is, for example, muscle wasting and attenuation of muscle wasting.

Muscle wasting is a loss of muscle mass due to reduced protein synthesis and/or accelerated breakdown of muscle proteins, largely as a result of activation of the non-lysosomal ATP-ubiquitin-dependent pathway of protein degradation. Muscle wasting is caused by a variety of conditions including cachexia associated with diseases including various types of cancer and AIDS, febrile infection, denervation atrophy, steroid therapy, surgery, trauma and any event or condition resulting in a negative nitrogen balance. Muscle wasting also occurs as a result of certain genetic conditions or mutations and following nerve injury, fasting, fever, acidosis and certain endocrinopathies.

Additional biological parameters include, for example, muscle contraction, muscle hypertrophy and muscle length. Further biological parameters include, for example, changes in gene expression after contact with a drug. In one aspect, the tissue/sensor combination described herein permits the assessment of changes in gene expression over time in response to a drug. Further, the effect of drugs on a tissue can be assessed while the tissue is mechanically challenged, e.g., placed under tension. Thus, unlike monolayer cultures, the tissues described herein permit the measurement of drug effects on tissues under differing mechanical stresses. The effects measured under different mechanical stresses can include, for example, mechanical effects, such as a change in contractile force, or biochemical changes, such as a change in gene expression. The ability to monitor gene expression under different mechanical stress conditions over time also permits the detection of changes in expression that occur independent of drug treatment. Thus, changes that occur over time in mechanically stressed tissues can reveal, for example, new drug targets.

In one aspect, a combination of direct and indirect measurement of biological parameters can be advantageous. For example, the direct measurement of contractile force using, for example micropost or birefringence techniques, can be performed in parallel with the measurement of gene activity using, for example, RT-PCR performed on tissue samples in adjacent wells or tubs that were exposed to the same agent. Using a plurality of similar tissues (e.g., on an array) permits one to directly analyze a biological parameter for one of the tissues over time following exposure to the agent, and to indirectly analyze another biological parameter by harvesting other members of the plurality at parallel time points for indirect analysis. In this way, different parameters can be monitored within the same experiment.

Use of Foreign DNA as a Marker for Screening Bioactive Compounds:

A tissue or organoid as described herein can produce a substance in an amount or of a type not normally produced by the cells or tissue in response to a bioactive compound (i.e. that can be measured, for example, a marker compound). In this aspect, at least some of the cells of the tissue or organoid contain a foreign DNA sequence. The foreign DNA sequence can be extrachromosomal, integrated into the genomic DNA of the tissue's cells, or can result from a mutation in the genomic DNA of the tissue's cells. In addition, the cells of the tissue or organoid can contain multiple foreign DNA sequences. Moreover, the different cells of the tissue or organoid can contain different foreign DNA sequences. For example, in one embodiment, a skeletal muscle tissue or organoid can include myofibers containing a first foreign DNA sequence and fibroblasts containing a second foreign DNA sequence. Alternatively, the skeletal muscle tissue or organoid could include myoblasts from different cell lines, each cell line expressing a foreign DNA sequence encoding a different marker compound. These “mosaic” tissues or organoids allow the combined and/or synergistic effects of particular bioactive compounds to be measured. For example, myoblasts expressing a detectable growth hormone coupled to a foreign DNA sequence of interest can be combined with myoblasts expressing green fluorescent protein or luciferase coupled to a foreign DNA sequence of interest to produce tissues or organoids expressing two detectable markers one secreted and, an additional marker, fluorescent or otherwise, of another cellular function.

In a preferred embodiment, the foreign DNA sequence encodes a protein which is sensitive to a bioactive compound or a substance that is measured as a biological parameter according to the invention. The protein is produced by the cells and liberated from the tissue or organoid. Alternatively, the DNA sequence can encode an enzyme or a cell surface protein which mediates sensitivity to a bioactive compound; or a detectable protein encoded by a reporter gene. The DNA sequence can also encode a DNA binding protein which regulates the transcription of the sequence responding to a bioactive compound or an anti-sense RNA which regulates translation of the mRNA responsive to a bioactive compound. The DNA sequence can also bind trans-acting factors, or direct the expression of a factor which can bind trans-acting factors, such that the transcription of the sequence (i.e., foreign or native) is responsive to a bioactive compound (e.g., by disinhibition). Furthermore, the foreign DNA sequence can be a cis-acting control element such as a promoter or an enhancer coupled to a native or foreign coding sequence responsive to a bioactive compound or for an enzyme which mediates the response to a bioactive compound. Thus, the foreign DNA sequence can be expressible in the cell type into which it is introduced and can encode a protein which is synthesized and which can be secreted by such cells. Alternatively, the foreign DNA sequence can be an element that regulates an expressible sequence in the cell. Alternatively, the foreign DNA sequence can encode for a receptor specific for certain classes of molecules or a ligand of a particular class of molecules, that is expressed at a level substantially above or below the normal, endogenous level of expression.

In Vitro Culture Conditions for Screening Assays:

Culture conditions for screening will vary according to the tissue produced. Methods for culturing cells are well known in the art and are described, for example, in Skeletal Cell Culture: A Practical Approach, (R. I. Fveshney, ed. IRL Press, 1986). The composition of the culture medium is varied, for example, according to the tissue produced, the necessity of controlling the proliferation or differentiation of some or all of the cells in the tissue, the length of the culture period and the requirement for particular constituents to mediate the production of a particular bioactive compound. The culture vessel can be constructed from a variety of materials in a variety of shapes as described.

As an example, for a varying period (e.g., 3 days) the cells can be maintained on growth medium containing DMEM-high glucose (GIBCO-BRL), 5% fetal calf serum (Hyclone Laboratories), and 1% penicillin/streptomycin solution (final concentration 100 units/ml and 0.1 mg/ml, respectively). The growth medium can be replaced manually or automatically by a perfusion system.

Micropost Arrays:

In one aspect, the sensor as described herein comprises one or more, and preferably two or more (e.g., 2, 3, 4, 10, 12, 20, 24, 48, 50, 96, 100, 192, 200, 384, 400, 500, 768, 800, 1000, 2000, 5000, etc.) microposts. Microposts are flexible rods of solid material that are attached to or surrounded by a tissue as described herein, and which provide a measure of, for example, the contractile force of a tissue through measurement of the distance between a micropost (or an end of a micropost) and a fixed reference point, or between the microposts or the ends of the microposts when, for example, two or more microposts are used.

Microposts can be employed in isotropic and anisotropic tissues. In one aspect, microposts are used with tissue that is anisotropic.

As noted herein above, the determination of bending deflections of microposts involves determining the stress distribution across the section of the micropost and using a model to determine the deflections of elastic beams (microposts). In one embodiement, muscle tissue surrounds a micropost and will ultimately deflect the post. In this situation the load from the muscle will be uniformly distributed along the post. The deflection of the post is describe by a well known formula used in solid mechanics [An Introduction to the Mechanics of Solids, Second Edition, S. H. Crandall, N. C. Dahl, and T. J. Lardner, 1978, McGraw-Hill Book Company]. The maximum deflection, δ_{MAx}, under a load wₒ, is given by the following expression:

$${δ}_{MAX} = \frac{{w}_{o} {L}^{4}}{8 EI}$$

where L is the length of the micropost, E is the elastic modulus, and I is the moment of inertia (for a cylinder the moment of intertia is a function of the radius) [An Introduction to the Mechanics of Solids, Second Edition, S. H. Crandall, N. C. Dahl, and T. J. Lardner, 1978, McGraw-Hill Book Company]. By calculating the moment of intertia of the post, knowing the elastic modulus of the micropost material (e.g., the polymer from which the posts were created using lithography), and knowing the length of the post, one can measure δ and then calculate the load (force).

The microposts can range from approximately 5 micrometers to 200 micrometers, most often approximately 5 to approximately 50 micrometers, depending on the length of the post and the elastic modulus of the polymer used in the process. The lengths of the post range from 10 micrometers to 250 micrometers. If the force from the muscle is small, then longer posts (L) and smaller radii posts are desirable to enhance the deflection. The figure below shows the parameters used in the calculation. The deflection can be measured under a microscope or with a CCD. The posts can waveguide light from the rear or they can be processed such that they have a fluorscent material on their tips.

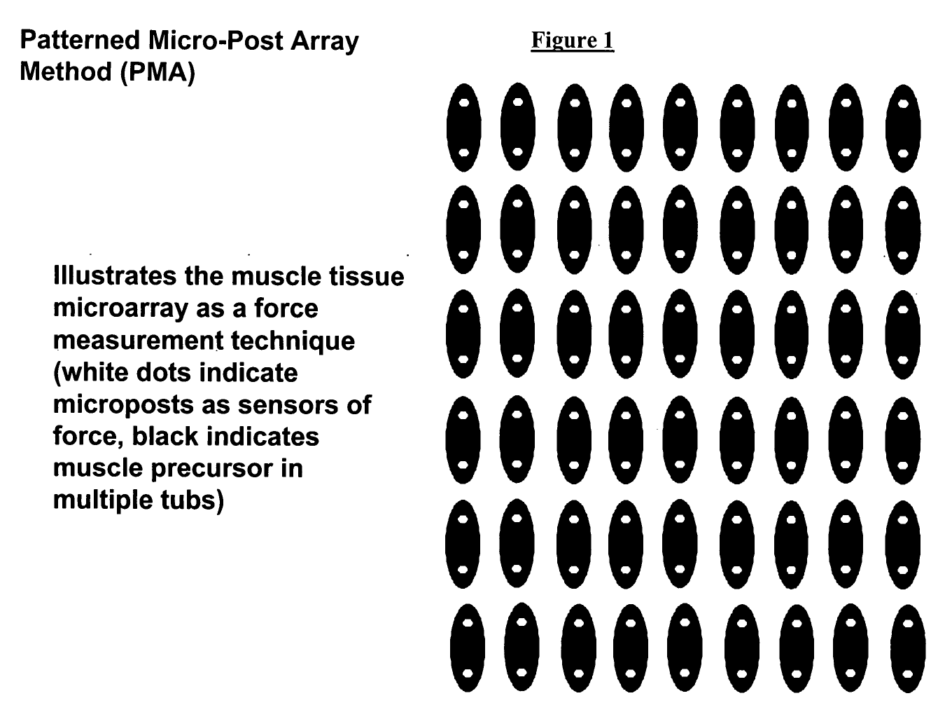

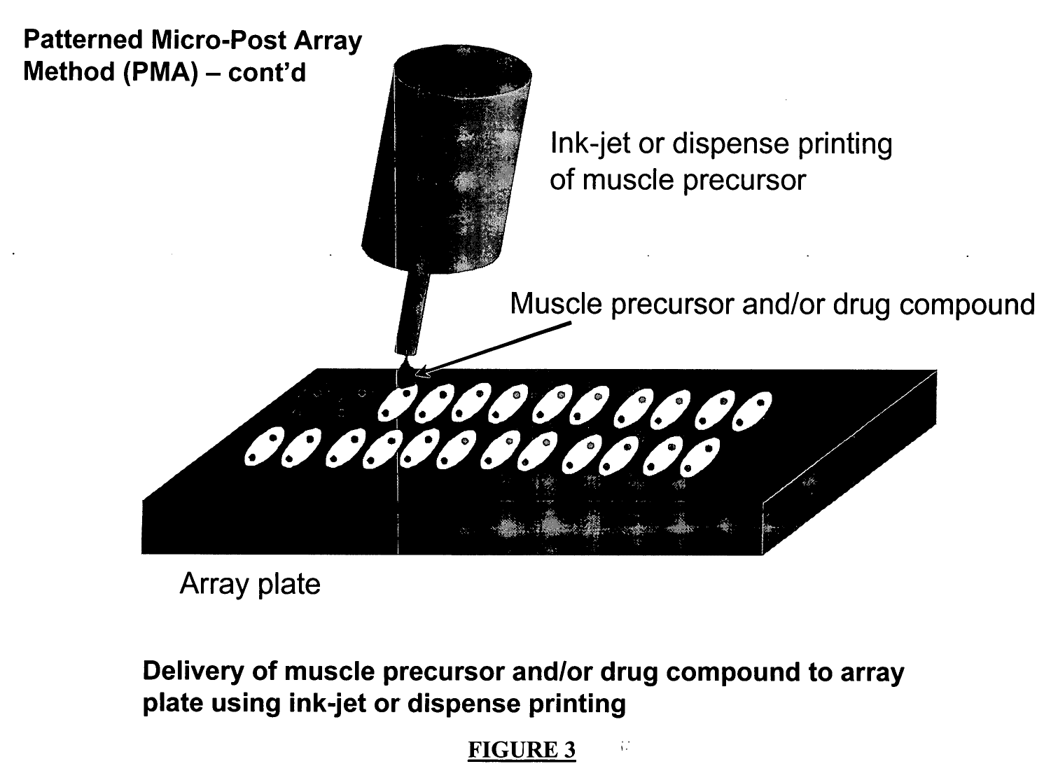

A micropost array (MPA) that permits muscle cells to grow anisotropically can be prepared through lithography or stamping. By confining muscle precursor materials to small ellipsoidal cells or “tubs” on the micrometer scale, the muscle can grow unidirectionally between two posts. Small muscle tubs have been created, with posts integrated into them which can be filled using inkjet printing or other micropipette techniques as shown, for example, in FIG. 3. A diagram of an array of such tubs comprising microposts is shown in FIG. 1. In the figure, which is a top view, the black area defines the tub or surface depressions in which the tissue is formed, and the microposts are shown in white.

MPAs can be made using wet lithography, using either positive or negative images (therefore positive or negative photoresist). For example, photoresists can be used or UV curable epoxies such as SU-8, an epoxy based negative resist can also be used. Cured SU-8 is highly resistant to solvents, acids, and bases, and it has excellent thermal stability; this epoxy is advantageous for using the cured structures as a permanent part of the device. Other ways to prepare such an MPA is to use the soft silicon rubber PDMS (polydimethylsiloxane) which can be filled into a microarray. This can be prepared using lithography or by creating a template in aluminan (for example) and filling it with PDMS. After PDMS is cured in the template, it can be peeled out. PDMS is a very soft and robust silicon rubber material used in all types of micro-stamping applications.

It is further contemplated that microposts can be positioned using electromagnets. In this aspect, the electromagnets could also facilitate the monitoring of the post positions.

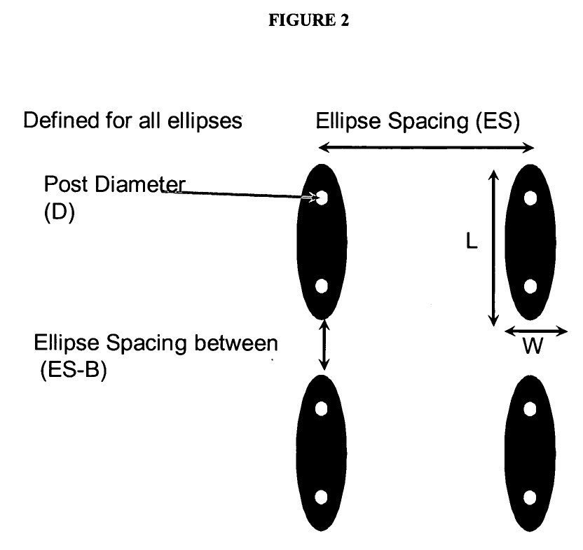

Dimensions of the micro-post array can vary for practical applications. Referring to FIG. 2, exemplary dimensions for ellipsoidal tubs are provided. The post diameter, D, can range, for example, from 5-200 micrometers, the length of the ellipse (major axis) can vary between 25-1000 micrometers, the width of the ellipse (minor axis) can vary between 25-1000 micrometers, the spacing between ellipses (between short axis) ES can be 25-1000 micrometers, and the spacing between ellipses (between long axis) ES-B can be 25-1000 micrometers. The thickness or height of the tub can be 25-500 micrometers. These values provide practical guidance but are not intended to be limiting.

In order to quickly and efficiently fill the tubs, an ink-jet or micro-pipette deposition can be used as shown in FIG. 3. Ellipsoidal or at least elongate tubs are preferred for muscle tissue. The muscle precursor is loaded into the syringe and accurately deposited into the ellipse tub. This is done in a sterile environment. The amount deposited will preferably exactly correspond to the tub volume. After deposition, the muscle tissue is nourished and grown in the array. Since the tub is anisotropic, it forces the muscle to grown unidirectionally. The anisotropic nature of the muscle actually enhances the strain on the posts, as compared to a muscle tissue that exerts isotropic strains on posts. This enables the strain to be greater and easier to measure. In addition, it can be measured more accurately because the force is essentially all along one direction, and because and it is a larger force on the posts owing to the anisotropic nature of the muscle tissue.

In addition to printing or pipetting into the tubs, the drug screening process can be performed in the very same way. Thus, after the muscle is grown, compounds for drug screening can also be delivered to the MPA by ink-jet or micro-pipette.

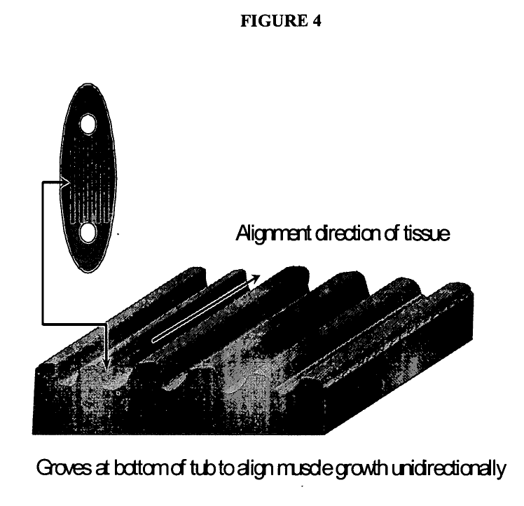

To enhance the muscle alignment in the tubs, corrugated surfaces can be created. This will assist the muscle precursor to align along the long axis of the ellipse. This can be created by performing the photolithography on a corrugated or grooved surface or can be created by lithography itself. This approach would provide alignment on the bottom surface in addition to the alignment introduced by the curvature of the ellipse. See FIG. 4.

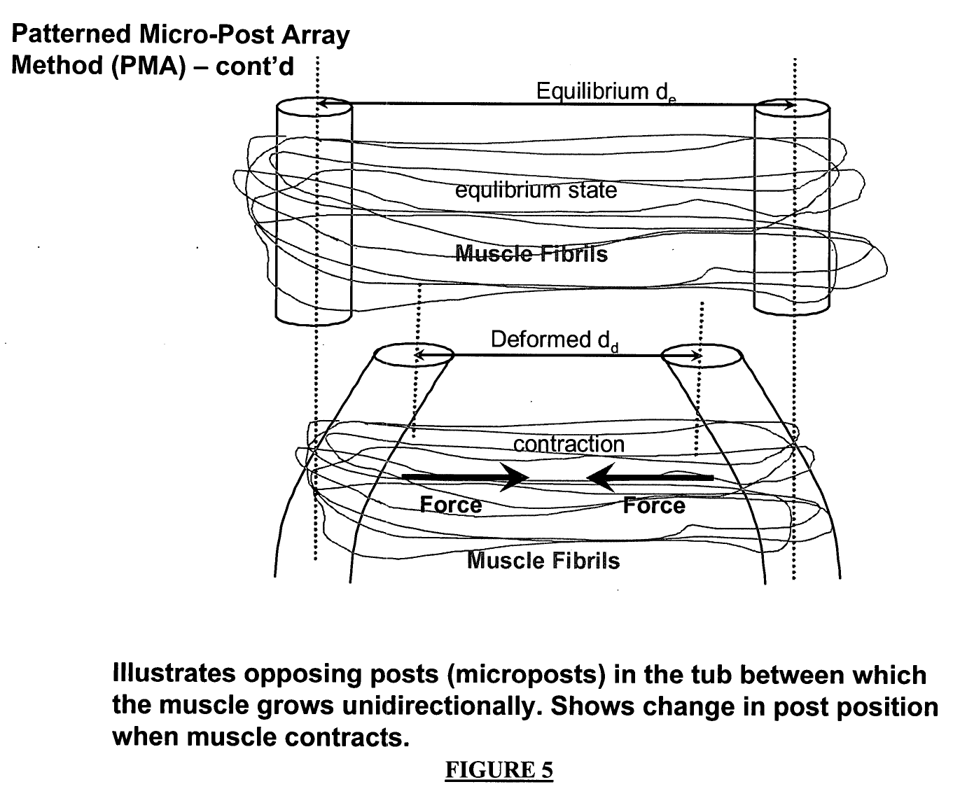

The process for measuring the force of, e.g., muscle contraction is straightforward. The posts in the tub will initially be in their equilibrium position (they may be straight or they may be bent a small amount due to inherent strain). Then a drug is applied, and the contraction of the muscle occurs along one direction. The contraction is amplified in comparison to isotropic contraction in the plane. The tips of the posts then point in and the new distance between them is measured and directly related to force. This is shown diagrammatically in FIG. 5. It is noted that if the posts are not fixed to the substrate, they will still move when the tissue contracts, also permitting measurement of contractile force.

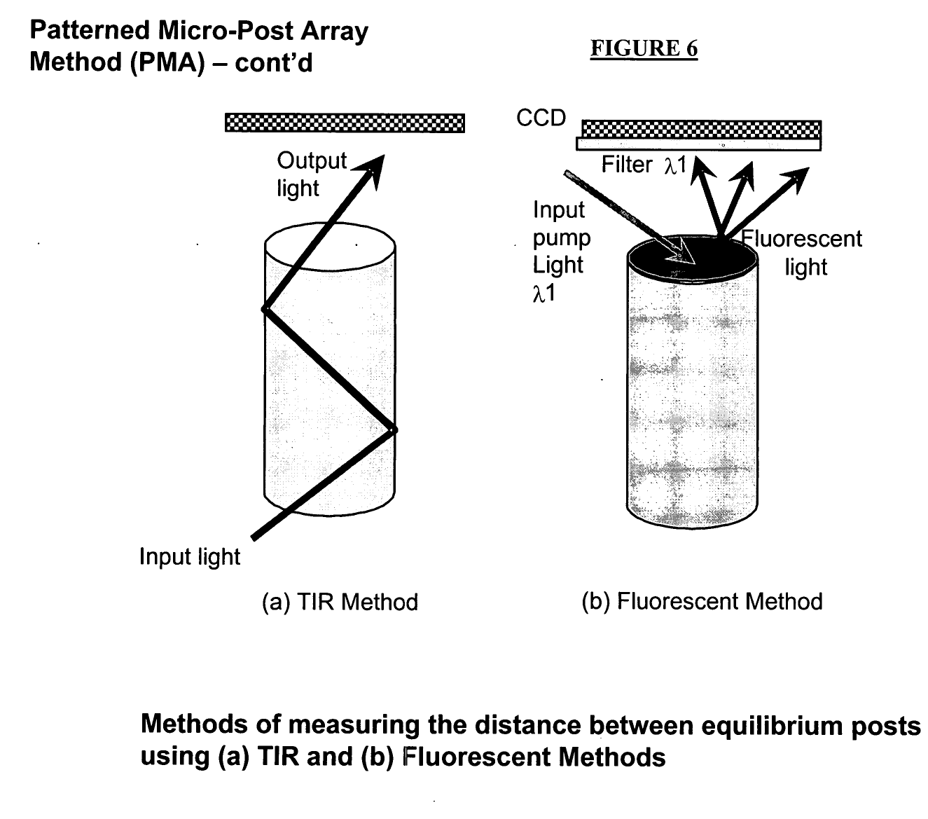

There are a number of ways to measure the distance between posts at equilibrium and the new distance after deformation. One can observe it directly under a microscope, or one can measure it with a CCD (charged coupled device) as shown, for example, in FIG. 6. At least two ways to measure the post position are possible: (1) Upon illumination from the bottom of the plate, the posts will capture the light of a given numerical aperture and there will be will total internal reflection (TIR) of the light through the posts—the output light is imaged on a CCD; or (2) the posts can have fluorescent materials on their tips so they can be front illuminated with a pump beam (for example UV light). The UV light is then converted to visible light which is visible with the CCD A filter beam would be used in the fluorescent case to block any residual pump light.

Additionally, to improve accuracy, one can perform drug screening in a number of wells or tubs for the same drug and average the force measurements. One can prepare different post sizes that will respond differently to different forces and average these for a single test. Other aspects providing for flexibility in the assay specifics will be apparent to the skilled artisan.

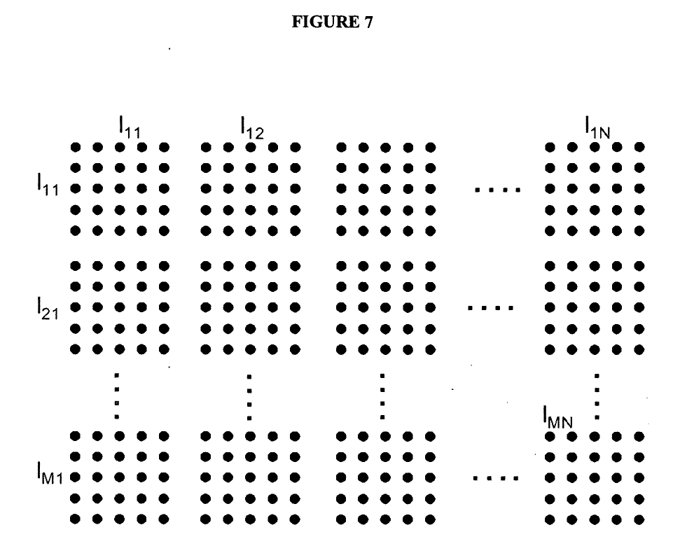

In one aspect, the micro-post array is a patterned micropost array, as shown, for example, in FIG. 7. In order to create arrays of micro-posts, the posts can be patterned on a surface (e.g., a slide or a plate, as described herein) or directly in multi-well arrays. These figures illustrate patterned micro-post arrays (MPA) on a planar substrate. Each grouping of posts is a single test bed for tissue (or cells). The post spacing, post diameter and the “lattice” arrangement of the posts can each be varied. FIG. 7 shows a simple square lattice. As used herein, the term “lattice unit cell” means one arrangement of posts that comprise the repeating unit of a lattice made up of such repeating units. Thus, for a square lattice, for example, a lattice unit cell is defined by the space between four posts set at the corners of a square. For a hexagonal lattice, for example, the lattice unit cell is defined by the space between six posts set at the corners of a regular hexagon.

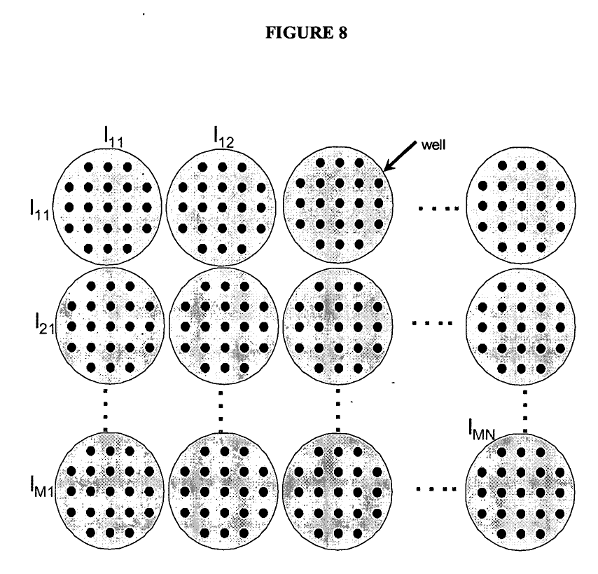

The MPAs can also be patterned in the bottom of standard well dishes (for example the 96 well dish). FIG. 8 shows a simple square lattice of posts integrated into the bottom of the wells. The post spacing, post diameter and the “lattice” arrangement of the posts can each be varied.

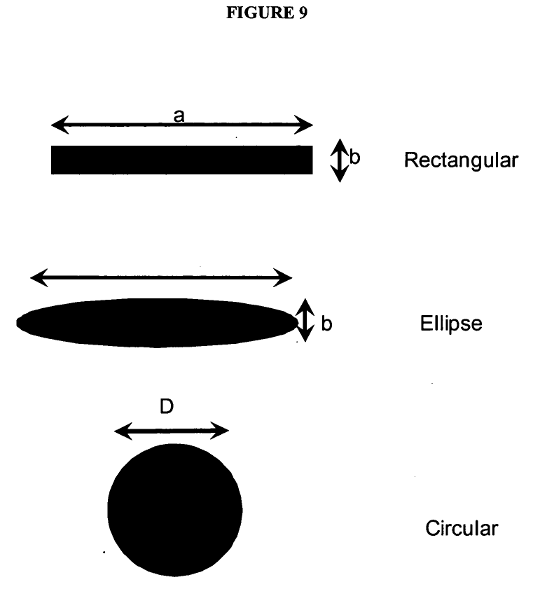

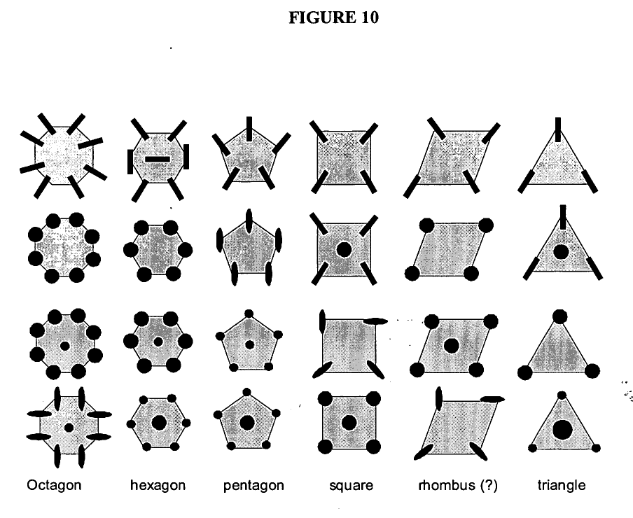

The post geometries can be varied for any embodiment employing a post. The most common post geometry is one with a circular cross section of diameter D (see FIG. 9). The diameter can be varied. For a given Young's modulus, the larger the post diameter, the less responsive it will be for a given load. Therefore the post diameter should be chosen to ensure that deflection will occur for a given load from the muscle tissue. Other post geometries can also be useful, such as those with rectangular (square) or ellipsoid cross sections (FIG. 9). They would be useful in determining the applied load along certain directions. For example the rectangular or ellipsoidal cross section (if a>>b as shown in the figure) would be more responsive to strains along the short (minor) axis b, and to a much lesser extent not responsive to strains along the long axis (major) a. If selectively patterned, one could in principle determine forces along two directions simultaneously. Furthermore, one may wish to use high aspect ratio (a/b) structures to better measure the average force along the short (minor axis).

Although simple lattices of posts may be most often used, other lattices can also be useful to measure anisotropy in force of muscles, or to map the force lines spatially exerted by the muscle. Several lattice unit cells are shown in FIG. 10, ranging from Octagon unit cells to triangular ones. Furthermore, depending on the application and needs, one may wish to mix various post diameters/shapes/aspect ratios to obtain the desired MPA for a given application. Here, the term “unit cells” is used loosely because they are often associated with orthogonal lattice configures (hexatic, square, triangular). However, other lattices types are not ruled out, such as the pentagon, which when patterned on a surface may only result in a quasi-lattice configuration. The unit cells illustrated in the figure are only examples of what is possible and are by no means limiting.

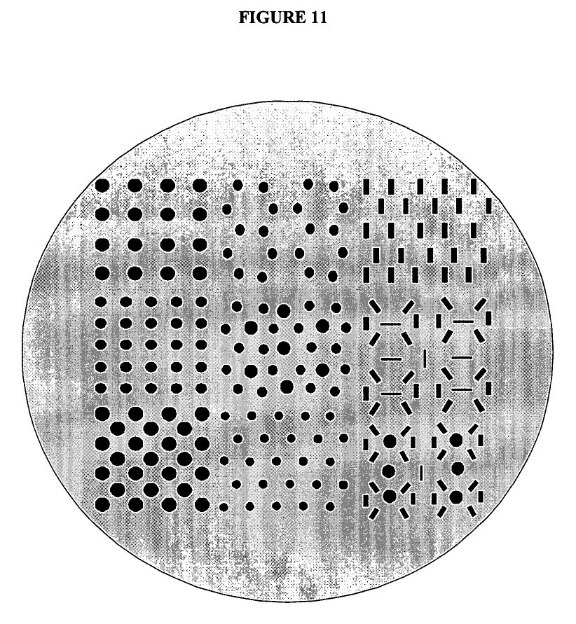

It may be useful for certain applications to pattern various arrays on the sub-well level as illustrated in FIG. 11. That is, there can be more than one unit cell arrangement within a given well, permitting the analysis of different tissue arrangements within the same well. There is no limitation to how one can pattern various post arrays, post sizes, and post geometries on a given well. If trying to determine anisotropic muscle interactions or probing various forces that may be unknown, it can be very useful, for example, to pattern an array in a manner that provides additional information. The patterned array in the figure is just one illustrative example of how this might be performed.

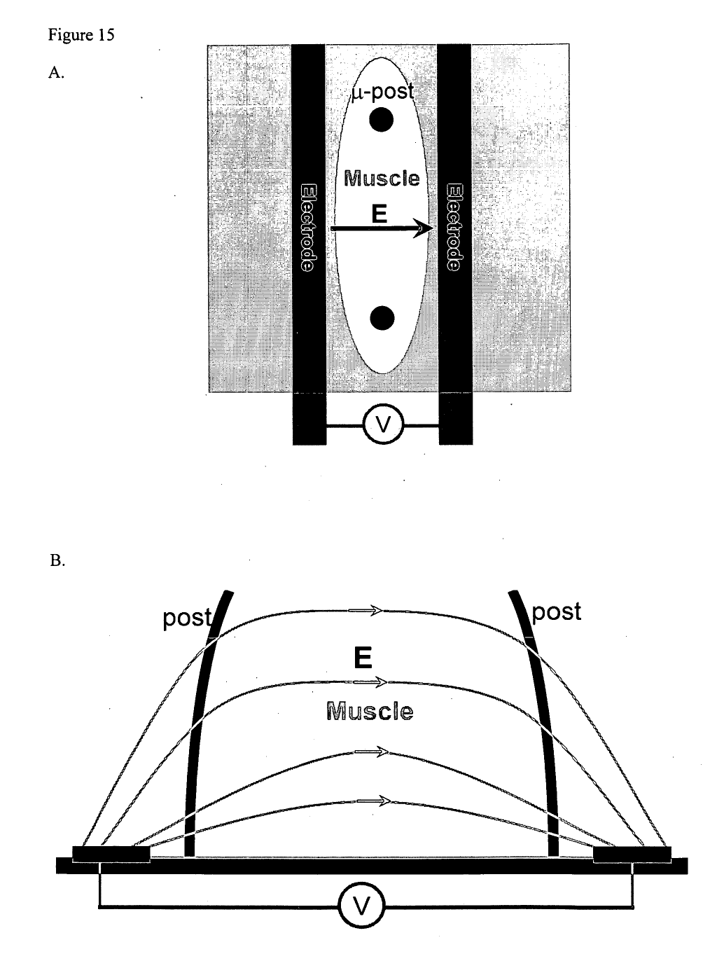

In another aspect, a plate or other tissue test substrate can be prepared such that electrodes are located on opposite ends or sides of the tissue, e.g., on opposite sides or ends of a tub, groove, or other arrangement comprising a tissue as described herein. The electrodes permit the application of an electrical field to the tissue. For muscle tissue, the electrical field can induce contraction or relaxation. This aspect can be combined, for example, with the micropost aspect to permit the monitoring or screening of drug effects on tissue function, e.g., contraction and relaxation. One embodiment of this tissue/sensor combination is shown schematically in FIG. 15, in which the electrodes flank an anisotropic tub comprising muscle tissue and two microposts.

Electrodes can be created in a number of ways, and the technique for the application of electrical fields to the tissue is not necessarily dependent upon the way in which the electrodes are constructed. In one embodiment, electrodes are created using wet lithography and indium-tin-oxide (ITO). A substrate with ITO coated over the entire surface is the starting point. Using positive photoresist, the electrodes are created using photolithography—i.e., a layer of photoresist is spin coated ontot he substrate, exposed with light through a mask with the in-plane electrodes, and subsequently etched, leaving behind only the in-plane electrodes on the substrate. Microposts and/or tubs can then be created lithography, such that they are registered between the electrodes, as shown, for example, in FIG. 15. When a voltage is applied across the electrodes, an electric field is created which can actuate the muscle. The posts deflection can then be measured to determine the force being exerted by the muscle tissue on the posts.

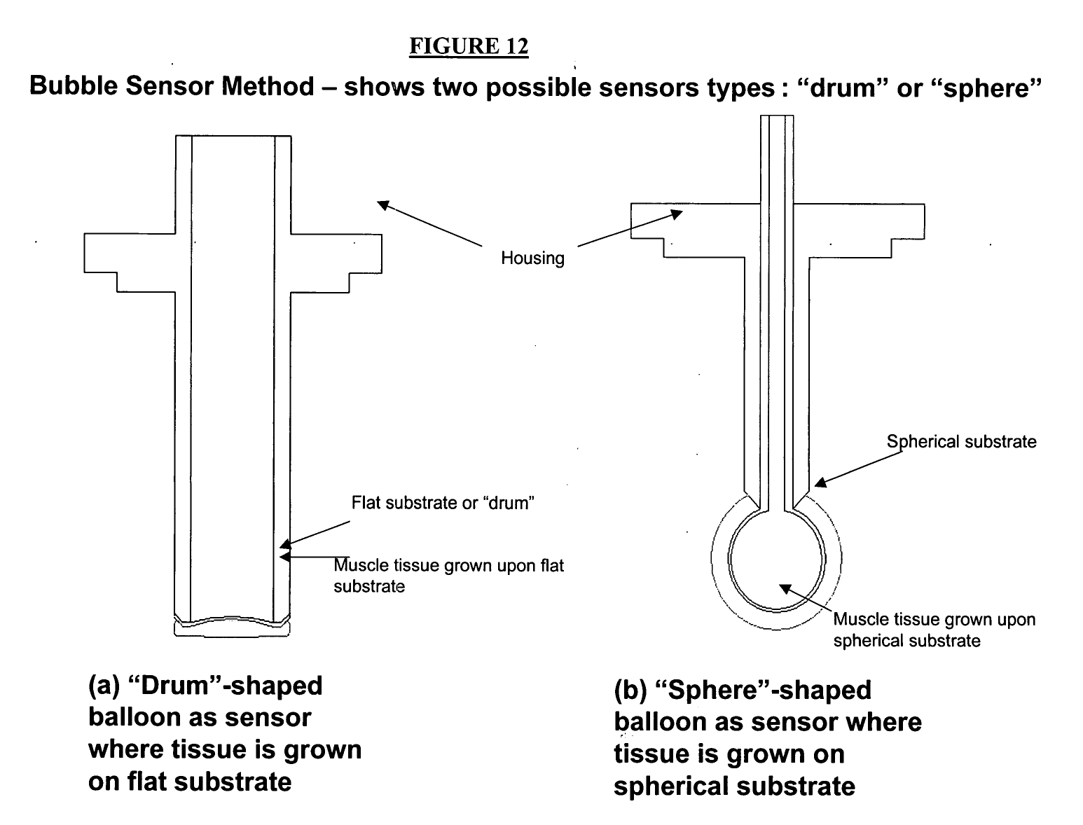

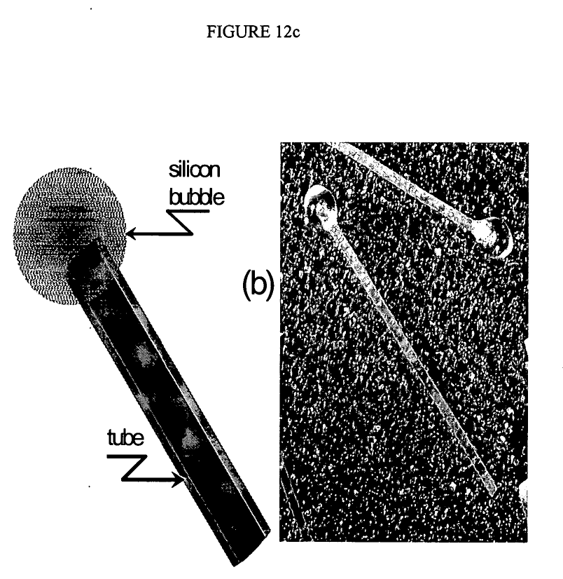

In another aspect, the tissue/sensor composition comprises tissue which is placed in contact with a sensor assembly comprising a sheet of elastic or pliable material covering or stretched over an opening, e.g., at a distal end of a hollow tube, similar to the way a skin is stretched over a drum head. The hollow tube can be, but is not necessarily, cylindrical, but should be hollow; e.g., a hollow square tube, a hollow rectangular tube, etc. It should also be understood that the tube can be, but is not necessarily, straight. The elastic material, in this “drum head” arrangement, is then placed in contact with the tissue. When contraction or relaxation of the tissue in response to a stimulus creates or removes a bulge in the tissue, this bulge generates a force on the drum head, which can then be measured, e.g., as a difference in pressure inside the tube. This aspect is diagrammed, for example, in FIG. 12a. In this aspect, the tissue is independent of the sensor.

In the aspect described above, pressure can be measured, e.g., using a pressure transducer as described herein. The hollow tube can be comprised of any material compatible with the environment of cell culture, e.g., glass or any of a number of polymers or plastics. In one embodiment, the tube is a capillary tube.

The elastic material can comprise, for example, an elastomer, silicon, polymer or another elastic material that is compatible with the tissue. The thickness of the elastic material can range between 1 μm to 150 μm or more, but may also include additional thicknesses. Sensitivity of this type of sensor construct depends, in part, upon the degree to which a given composition and thickness of the elastic material is able to flex in response to a change in the tissue and thereby create a change in pressure inside the tube. Generally, thinner sheets of elastic material will be more sensitive. However, thinner sheets of elastic material will also be more susceptible to damage than thicker ones. These considerations can be used by one of skill in the art to adapt this sensor design to a given tissue arrangement.

For the drum-head-like sensor aspect, the tissue can be grown on an exterior surface of the drum head. Alternatively, the tissue can be prepared separate from the sensor, with the sensor being placed in contact with the tissue after the tissue is formed.

Drum head assemblies as described above can be used singly, e.g., where one assembly is contacted with a plurality of separate tissues. Alternatively, the drum head assemblies can be arranged in an array. In one embodiment, the array corresponds to an array of tissues, e.g., as described herein, such that an array of tissues can be monitored by the drum head sensors at the same time. In another embodiment, drum head assemblies can be arranged in, on or over a plate arrangement as described herein.

In another aspect, a similar pressure-sensing approach is used, but the tissue is grown or deposited around the outside of a compressible “bubble” of elastic material extending from a distal end of a hollow tube (again, the tube need not be cylindrical, but should be hollow). By “compressible” is meant that the bubble of elastic material yields to pressure from outside, such as the pressure created when muscle tissue on its outer surface contracts.

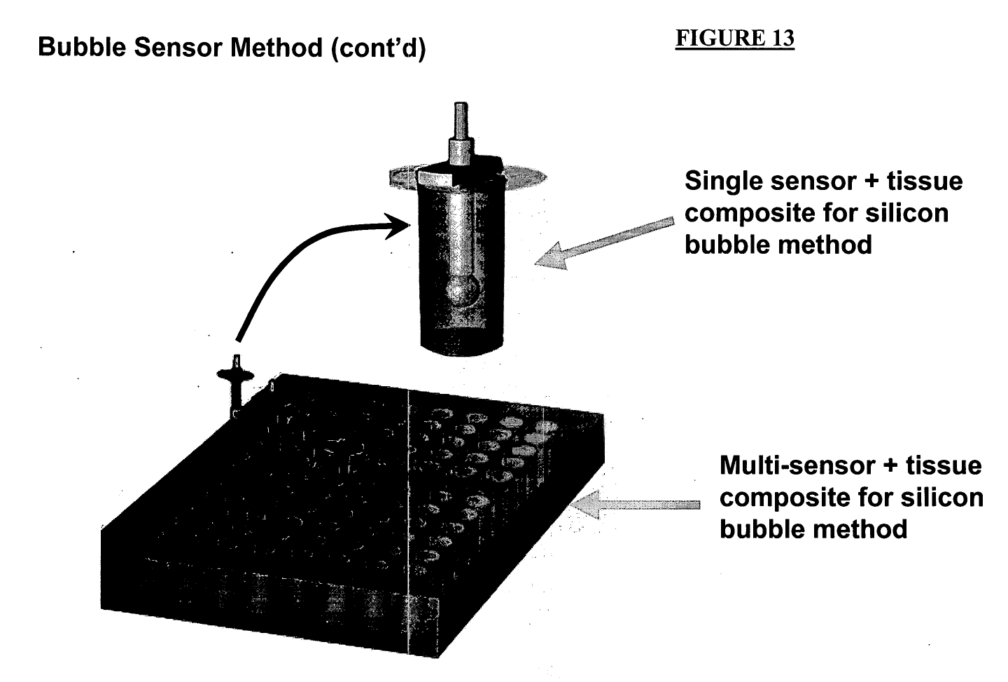

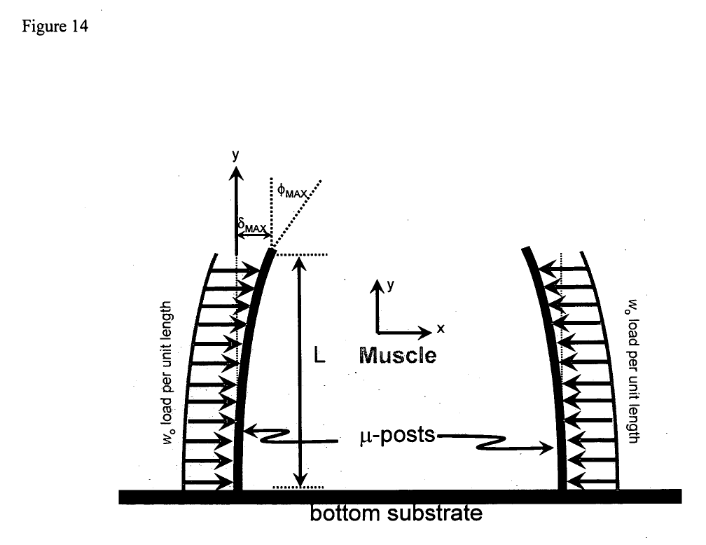

For this aspect, the elastic material can comprise materials and thicknesses as described above in relation to the drum head sensor assembly. The shape of the bubble is not critical and can be, for example, oval, elliptical, polygonal (e.g., pyramidal, cubic, hexagonal, etc.). An approximately spherical shape is preferred. Contraction of the muscle tissue on this sensor assembly generates a force on the bubble that can be measured, e.g., as a change in the pressure inside the bubble (and the hollow member from which it extends). Changes in pressure in the tube are detected, e.g., with a pressure transducer as described herein. An embodiment of this aspect is diagrammed in FIG. 12b. In this aspect, the tissue is not independent from the sensor. In this aspect and in the “drum head” aspect, the sensor assembly can be arranged, for example in a housing member that supports the sensor. An array of such sensor assemblies arranged, e.g., to correspond to an array of tissue tubs, e.g., as described above in relation to the micropost array, can also be used to monitor a plurality of tissues (see, e.g., FIG. 13). Alternatively, such sensor assemblies are arranged in, on or over a plate as described herein.

Compounds of Use in the Methods Described:

The term “compound” refers to a chemical compound (naturally occurring or non-naturally occurring), such as a synthetic drug, small molecule, biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. Compounds are evaluated for potential activity as inhibitors or activators (directly or indirectly) of a biological process or processes (e.g., agonist, partial antagonist, partial agonist, antagonist, antineoplastic agents, cytotoxic agents, inhibitors of cell proliferation, cell proliferation-promoting agents, and the like) by inclusion in screening assays as described herein. The activities (or activity) of a compound can be known, unknown or partially-known. The compound can be administered orally, through an injection or using other means. Such compounds can be screened for activity using the methods described herein.

The term “compound” further refers to a compound to be tested by one or more screening method(s) as a putative modulator. Usually, various predetermined concentrations are used for screening such as 0.01 μM, 0.1 μM, 1.0 μM, and 10.0 μM, but can range from, for example, about 0.01 nM to about 10 mM. Test compound controls can include the measurement of a bioactivity in the absence of the test compound or comparison to a compound known to increase or decrease a bioactivity of interest.

Bioactive compounds of interest include, but are not limited to, for example, synthetic drugs (including, for example, small molecules), bioactive proteins, receptors, enzymes, ligands, regulatory factors, and structural proteins. Nuclear proteins, cytoplasmic proteins, mitochondrial proteins, secreted proteins, plasmalemma-associated proteins, serum proteins, viral antigens and proteins, bacterial antigens, protozoal antigens and parasitic antigens are also useful according to the invention.

As used herein, “bioactivity” includes but is not limited to a bioactivity performed by a tissue as described herein, for example, muscle contraction, muscle lengthening or shortening, muscle hypertrophy, mRNA or protein synthesis.

The methods described herein can be used to identify compounds that increase or decrease bioactivity, for example, muscle contraction or relaxation of a tissue of the invention. For example, the invention provides for methods of identifying compounds including but not limited to gastrointestinal stimulants, antihypertensive agents, smooth muscle relaxants, bladder antispasmodic compounds, urinary bladder contraction medication compounds, muscarinic blocking compounds, compounds that increase or decrease constipation, compounds that increase or decrease the activity of ACE inhibitors, antihypertensive agents, post myocardial infarction compounds, compounds that prevent heart failure and antiarrhytmic compounds.

The methods as described herein and compounds identified by these methods can also be used to induce a contraction in a tissue of interest. The methods described herein can be used to detect gene level changes in the presence or absence of added compounds and or static or active mechanical conditions.

The invention also provides for methods of measuring the permeability of a compound that increases or decreases a property, as defined herein, of a tissue as described herein. The measurement of permeability can be performed, for example, by positioning a tissue between two chambers of a culture dish, such that a molecule can only pass from one chamber to the other by passing through the tissue. The sensor in this embodiment measures the amount or presence of the molecule in one or both chambers. Both the rate and extent of passage of the molecule can be measured.

Kits:

Tissue-containing kits are also useful in the methods described herein. For example, a kit that includes a plurality (i.e., at least 6, preferably 24, 48, 96, and even up to several thousand) of tissues (e.g., organized tissues) individually contained in a container that permits culture conditions in which the organized tissue is viable long term is particularly useful according to the invention. Minimally, the container will contain physiological medium that permits viability of the tissue for storage and/or shipment purposes. Desirably, the medium and container will permit long-term viability and detection of a biological parameter of the tissue as described herein.

“Physiological” medium refers to any physiological solution of salts and nutrients that permits maintenance of the tissue for at least 15 days, and shipment of the organized tissue; for example a medium for long term viability of the tissue can consist of DMEM with high glucose, 10% horse serum, 5% fetal calf serum, and 100 units/ml penicillin.

Use and Administration:

Candidate bioactive compounds identified using the methods described herein are potentially useful in treating disease involving a given tissue. Such compounds, once identified and tested for efficacy, can be delivered systemically or locally to an organism by a wide variety of methods. For example, an exogenous source (i.e. produced outside the organism treated) of the bioactive compound may be provided intermittently by repeated doses. For treatment, the route of administration can include oral consumption, injection, or tissue absorption via topical compositions, suppositories, inhalants, or the like. Exogenous sources of the bioactive compound can also be provided continuously over a defined time period. For example delivery systems such as pumps, time-released compositions, or the like can be implanted into the organism on a semi-permanent basis for the administration of bioactive compounds (e.g. insulin, estrogen, progesterone, etc.). Efficacy of the compound in disease treatment is indicated by amelioration or prevention of disease symptoms or the disease itself. The methods and compositions described herein can also be used for screening potential biological and chemical toxins.

### EXAMPLES

### Example 1

### Preparation of a Tissue in Combination with a Sensor

A tissue in combination with a sensor can be prepared as follows, which exemplifies a preparation using muscle tissue.

To produce skeletal muscle organoids, primary avian, rat or human muscle stem cells or immortalized murine muscle cells, were suspended in a solution of collagen and Matrigel™ which was maintained at 4° C. to prevent gelling. The cell suspension was then placed in a vessel with tissue attachment surfaces coupled to an interior surface at each end of the vessel. The vessel was positioned in the bottom of a standard cell culture chamber. Following two to four hours of incubation at 37° C., the gelled cell suspension was covered with fresh culture medium (renewed at 24 to 72 hour intervals) and the chamber containing the suspended cells was maintained in a humidified 5% CO₂ incubator at 37° C. throughout the experiment.

Between the second and sixth day of culture, the cells were found to be organized to the extent that they spontaneously detached from the vessel. At this stage, the cells were suspended in culture medium while coupled under tension between tissue attachment surfaces positioned at either end of the culture vessel. During the subsequent ten to fourteen days, the cells formed an organoid containing skeletal myofibers aligned parallel to each other in three dimensions. The alignment of the myofibers and the gross and cellular morphology of the organoid were similar to that of in vivo skeletal muscle.

To carry out the above method, an apparatus for organoid formation was constructed from silastic tubing and either VELCRO™ or metal screens as follows. A section of silastic tubing (approximately 5 mm I.D., 8 mm O.D., and 30 mm length) was split in half with a razor blade and sealed at each end with silicone rubber caulking. Strips of VELCRO™ (loop or hook side, 3 mm wide by 4 mm long) or L-shaped strips of stainless steel screen (3 mm wide by 4 mm long by 4 mm high) were then attached with silicone rubber caulking to the interior surface of the split tubing near the sealed ends. The apparatus was thoroughly rinsed with distilled/deionized water and subjected to gas sterilization.

Skeletal muscle organoids were produced in vitro from a C2C 12 mouse skeletal muscle myoblast cell line stably co-transfected with recombinant human growth hormone-expressing and β-galactosidase-expressing (β-gal) constructs (Dhawan et al., 1991, Science 254:1509-1512) or from primary avian myoblasts or from primary rat myoblasts (both neonatal and adult cells) or from primary human myoblasts (both fetal and adult satellite cells).

Cells were plated in the vessel at a density of 1-4×10⁶ cells per vessel in 400 μl of a solution containing extracellular matrix components. The suspension of cells and extracellular matrix components was achieved by the following method. The solution includes 1 part Matrigel™ (Collaborative Research, Catalog No. 40234) and 6 parts of a 1.6 mg/ml solution of rat tail Type I collagen (Collaborative Research, Catalog No. 40236). The Matrigel™ was thawed slowly on ice and kept chilled until use. The collagen solution was prepared just prior to cell plating by adding to lyophilized collagen, growth medium (see constituents below), and 0.1N NaOH in volumes equivalent to 90% and 10%, respectively, of the volume required to obtain a final concentration of 1.6 mg/ml and a pH of 7.0-7.3. The collagen, sodium hydroxide and growth medium were maintained on ice prior to and after mixing by inversion.

Freshly centrifuged cells were suspended in the collagen solution by trituration with a chilled sterile pipet. Matrigel™ was subsequently added with a chilled pipet and the suspension was once again mixed by trituration. The suspension of cells and extracellular matrix components was maintained on ice until it was plated in the vessel using chilled pipet tips. The solution was pipetted and spread along the length of the vessel, taking care to integrate the solution into the tissue attachment surfaces. The culture chamber containing the vessel was then placed in a standard cell culture incubator, taking care not to shake or disturb the suspension. The suspension was allowed to gel, and after 2 hours the culture chamber was filled with growth medium such that the vessel was submerged.

Skeletal muscle organoids were produced from adult human biopsied skeletal muscle by the following method. Standard muscle biopsies were performed on two adult male volunteers and myoblasts isolated by standard tissue culture techniques (Webster et al., 1990, Somatic Cell and Mol. Gen. 16:557-565). One hundred muscle stem cells (myoblasts) were identified from each biopsy by immunocytochemical staining with an antibody against desmin and the myoblasts were expanded through at least 30 doubling. The 100 myoblasts could thus be expanded into greater than 50 billion cells (5×10¹⁰).

Skeletal muscle cells were cultured into organoids according to the following conditions. For a period of three days' the cells were maintained on growth medium containing DMEM-high glucose (GIBCO-BRL), 5% fetal calf serum (Hyclone Laboratories), and 1% penicillin/streptomycin solution (final concentration 100 units/ml and 0.1 mg/ml, respectively). On the fourth day of culture, the cells were switched to fusion medium containing DMEM-high glucose, 2% horse serum (Hyclone Laboratories), and 100 units/ml penicillin for a period of 4 days. On the eighth day of culture, the cells were switched to maintenance medium containing DMEM-high glucose, 10% horse serum, 5% fetal calf serum, and 100 units/ml penicillin for the remainder of the experiment. In certain embodiments cells were maintained in a defined serum-free medium containing insulin, transferrin and selenium. Before the organoids were ready for implantation, some were cultured in maintenance media containing 1 mg/ml of cytosine arabinoside for the final four to eight days. Treatment with cytosine arabinoside eliminated proliferating cells and produced organoids containing substantially post-mitotic cells. The growth medium can be replaced manually or automatically by a perfusion system.

Sensors are introduced to the tissue either during or after the formation of the tissue. Where the sensor is introduced during the formation, for example, the cells and matrix material are deposited between the ends of a differential force transducer, to which the muscle fibrils attach. Alternatively, the differential force transducer is connected to either end of a tissue after the formation of the tissue, e.g., as when a probe or probes connected to the transducer are inserted into the tissue.

Alternatively, tissue can be prepared by depositing a suspension of dissociated muscle cells and extracellular matrix components into an array of tubs in a substrate, e.g., a substrate comprising an array of tubs comprising microposts, prepared, e.g., by wet lithography. In one approach, an inkjet printer head is used to deposit the suspension of cells into receptacles. After the cell suspension is deposited on the array, it is then incubated in the presence of culture medium. The muscle cells become arranged into small tissues along the elongate axis of the tubs. By virtue of their having surrounded the microposts at the ends of the wells during the formation of the tissues, the tissues are arranged between the microposts. Contraction or relaxation of the tissues in response to a test compound can then be measured by monitoring changes in the distances between the microposts (or their ends).

Alternatively, tissue-sensor assemblies can be formed by suspending a hollow tube with a bubble of elastic material in a well containing a suspension of dissociated muscle cells and matrix material. When incubated under cell culture conditions, the muscle cells attach to the exterior of the bubble and form a tissue. Connection of the hollow tube to a pressure transducer permits measurement of the contractile state of the muscle tissue.

Tissues can be prepared on any suitable substrate in any arrangement. For example, cells and matrix components can be deposited onto a plate or substrate in a desired pattern, to form, e.g., an array of tissues, or into a well of a multi-well dish or into individual tubs within a plate or well.

### Example 2

### Use of a Tissue Sensor for Screening a Compound for Bioactivity

A tissue and sensor, e.g., a muscle tissue and sensor prepared as described herein can be used to screen for bioactive compounds, for example, as follows.

An array of tissues formed in isolated wells in, e.g., a 384 or 96 well tissue culture dish is contacted with test compound by adding the test compound(s) to the wells, either individually or with a multipipettor. Readings from the sensor before and after (e.g., 1 second, 5 seconds, 30 seconds, 1 min, 5 min, 1 hr, etc. after) addition of the test compound determine the bioactivity of the compound. For example, contraction or relaxation of muscle tissue occurring after introduction of the compound is indicative that the compound is bioactive on the tissue.

The method of performing the assay will vary depending upon the particular assembly of the sensor and tissue. For example, where the tissue is independent from the sensor, a single sensor can be sequentially placed in contact with a plurality of tissues, each, for example, contacted with a different compound. Alternatively, where the sensor is not independent of the tissue, the sensor will remain in contact with one tissue throughout the assay, and an array of sensor/tissue assemblies can be used to screen more than one compound or more than one dosage of compound. For these and other assays, compounds can be tested in ranges of concentration varying from, e.g., about 0.01 nM to about 10 mM. The readings from the sensor(s) can be viewed as, for example, changes on an oscilloscope, ammeter, pressure transducer, or optical detector.

### Example 3

### Use of a Tissue Sensor for Screening a Library of Compounds for Bioactivity

A library of compounds is screened by, for example, preparing an array of tissues in combination with a sensor, and then contacting different members of the array with different members of the library of compounds. This can be achieved, for example, where a library of compounds is added to different wells of an array of wells comprising tissue. The tissue can be in combination with a sensor within the well, or a sensor or set of sensors can be moved from well to well, depending upon the type of sensor used.

In one aspect, an array of bubble-type sensor-muscle tissue assemblies is immersed in wells of a plate comprising members of the library. Pressure readings from the array of sensor-tissue assemblies provides a read out on the bioactivity of members of the library. In one embodiment, which is applicable to any of the library screening approaches described herein, the library is split up into a number of wells, each well comprising a subset of the library's members. When a well with a given subset is found to have a desirable effect, the subset is then further separated into a number of separate wells, and the process repeated until an individual member of the library is identified that has the desired activity.

Alternatively, members of a library of compounds can be dispensed into tubs containing muscle tissue-micropost assemblies as described herein. The contractile state of the tissues is monitored by, for example, monitoring the distance between posts using, e.g., the TIR method or the fluorescent method as described herein.

In this manner, compounds that induce contraction of muscle tissue can be identified where the test compound causes a decrease in the distance between microposts. Compounds that induce a relaxation of muscle tissue can also be identified, for example, if the tissue is treated with a known inducer of contraction prior to addition of the test compound—relaxation of contracted tissue is evident from an increase in the distance between microposts.

### Other Embodiments

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A composition comprising a container comprising at least one viable tissue in combination with a sensor, wherein said tissue was formed in vitro.

2. The composition of claim 1 wherein said tissue is independent of said sensor.

3. The composition of claim 1, wherein said tissue is not independent from said sensor.

4. The composition of claim 1, wherein said tissue comprises muscle cells.

5. The composition of claim 3, wherein said muscle cells are selected from the group consisting of: smooth, skeletal or cardiac muscle cells.

6. The composition of claim 1, wherein said tissue is organized.

7. The composition of claim 1, wherein said sensor measures an optical, physical, chemical, genetic or electrical property of said tissue.

8. The composition of claim 1, wherein said sensor measures at least one of muscle contraction, muscle relaxation, muscle hypertrophy and muscle length.

9. The composition of claim 1, further comprising a device to provide a readout for a change in a property of said tissue.

10. A plate comprising at least one tissue in combination with a sensor, wherein said tissue was formed in vitro.

11. The plate of claim 10, wherein said sensor is independent from said tissue.

12. The plate of claim 10, wherein said tissue is not independent from said sensor.

13. The plate of claim 10 wherein said plate comprises at least two microposts.

14. The plate of claim 13 wherein said microposts are attached to said plate.

15. The plate of claim 13 wherein said microposts are supported by an extracellular matrix material.

16. The plate of claim 15 wherein said extracellular matrix material comprises collagen.

17. The plate of claim 10 wherein said tissue is in contact with two or more microposts.

18. The plate of claim 13 that comprises an array of microposts.

19. The plate of claim 18 wherein said array comprises one or more lattice unit cells defined by the arrangement of said microposts.

20. The plate of claim 10 which comprises a plurality of wells that comprise said tissue.

21. The plate of claim 20 wherein a well of said plurality of wells comprises at least two microposts.

22. The plate of claim 21 wherein a well of said plurality of wells comprises an array of microposts.

23. The plate of claim 22 wherein a said array comprises one or more lattice unit cells defined by the arrangement of said microposts.

24. The plate of claim 13 wherein said tissue is in contact with at least two of said microposts.

25. The plate of claim 24 wherein said tissue is in contact with and located between at least two of said microposts.

26. The plate of claim 22 wherein a said tissue is in contact with and located between a plurality of pairs of the microposts in said array.

27. The plate of claim 23 wherein a said tissue is in contact with and located between each micropost defining a lattice unit cell.

28. The plate of claim 10, wherein said tissue comprises muscle cells.

29. The plate of claim 10, wherein said muscle cells are selected from the group consisting of: smooth, skeletal or cardiac muscle cells.

30. The plate of claim 10, wherein said tissue is organized.

31. The plate of claim 10 which comprises one or more essentially linear grooves.

32. The plate of claim 31 wherein said one or more essentially linear grooves are located in one or more wells on said plate.

33. The plate of claim 31 wherein said grooves are arranged substantially parallel to each other.

34. The plate of claim 31 wherein said tissue is arranged in said one or more grooves.

35. The plate of claim 31 wherein one or more of said grooves comprise at least two microposts.

36. The plate of claim 35 wherein said tissue is in contact with and located between at least two of said microposts.

37. The plate of claim 13, wherein said sensor measures a change in the distance between said microposts.

38. The plate of claim 10, wherein said sensor measures at least one of muscle contraction, muscle relaxation, muscle hypertrophy, and muscle length.

39. The plate of claim 10, further comprising a device to provide a readout for a change in a property of said tissue.

40. An array comprising at least one tissue in combination with a sensor, wherein said tissue was formed in vitro.

41. The array of claim 40, wherein said sensor is independent from said tissue.

42. The array of claim 40, wherein said tissue is not independent from said sensor.

43. The array of claim 40, wherein said tissue comprises muscle cells.

44. The array of claim 40, wherein said muscle cells are selected from the group consisting of: smooth, skeletal or cardiac cells.

45. The array of claim 40, wherein said tissue is organized.

46. The array of claim 40, wherein said sensor is optical, physical, electrical or chemical.

47. The array of claim 40, wherein said sensor measures at least one of muscle contraction, muscle relaxation, muscle hypertrophy, and muscle length.

48. The array of claim 40 comprising a plurality of microposts.

49. The array of claim 48 wherein said tissue is in contact with and located between at least two of said microposts.

50. The array of claim 40, further comprising a device to provide a readout for a change in a property of said tissue.

51. An apparatus comprising at least a tissue formed in vitro, in combination with:
a) a sensor; and
b) a device that provides a readout for a change in a property of the tissue.

52. A method of screening a compound for bioactivity, comprising contacting a candidate bioactive compound with a tissue, wherein said tissue is in combination with a sensor, and measuring in said tissue a biological parameter that is associated with bioactivity, wherein a change in the biological parameter that occurs as a result of said contacting step is indicative of bioactivity of said candidate compound.

53. A method of screening a library of compounds for bioactivity, comprising contacting a candidate bioactive compound from said library with a tissue, wherein said tissue is in combination with a sensor, and measuring in a tissue a biological parameter that is associated with bioactivity, wherein a change in the biological parameter that occurs as a result of said contacting step is indicative of bioactivity of said candidate compound.

54. A method of identifying a compound that increases or decreases muscle contraction or muscle relaxation comprising contacting a candidate compound with a tissue, wherein said tissue is in combination with a sensor, and measuring in said tissue, muscle contraction, wherein an increase or decrease in muscle contraction that occurs as a result of said contacting step is indicative of said compound modulating muscle contraction.

55. A method of monitoring the effect of an agent on a tissue, the method comprising the steps of:
a) providing a plurality of tissues formed in vitro, wherein at least one of said tissues is in combination with a sensor;
b) contacting said plurality of tissues with an agent;
c) obtaining a measurement from said sensor; and
d) detecting a nucleic acid sequence in a said tissue, wherein an effect of said agent on said tissues is determined.

56. The method of claim 55 wherein the step of detecting a nucleic acid sequence in a said tissue comprises isolating nucleic acid from a tissue of said plurality.

57. The method of claim 55 wherein the step of detecting a nucleic acid sequence in a said tissue comprises amplification of a nucleic acid sequence from a tissue of said plurality.

58. The method of claim 55 wherein the step of detecting a nucleic acid sequence in a said tissue comprises hybridization of nucleic acid prepared from said tissue to an array.

59. The method of claim 55 wherein the step of detecting a nucleic acid sequence in a said tissue comprises obtaining a genetic expression profile for said tissue.

60. The method of claim 55 wherein said contacting step is repeated at least once.

61. The method of claim 55 wherein steps (c) and (d) are repeated at least once.

62. The method of claim 61 wherein said steps of detecting detect a change in the genetic expression profile for said tissues.

63. The method of claim 55 wherein said tissue is prepared from cells from an individual having a condition affecting said tissue.

64. The method of claim 55 wherein said tissue comprises a genetically modified cell.

65. A method of inducing muscle contraction or muscle relaxation in a tissue in combination with a sensor, wherein said tissue is contacted with a compound, a mechanical force or an electrical force.

66. A method of measuring permeability of a compound that increases or decreases at least one of muscle contraction, muscle relaxation, muscle hypertrophy, muscle mass and muscle length, comprising introducing said compound into a sensor, wherein said sensor is in combination with a tissue, and wherein said permeability is measured by determining a change in at least one of muscle contraction, muscle hypertrophy, muscle mass and muscle length of said tissue.

67. The method of claim 52 or 53, wherein said biological parameter is selected from the group consisting of: muscle contraction, muscle relaxation, muscle hypertrophy, muscle length, gene expression, mRNA expression, protein expression, enzymatic activity.

68. The method of any one of claims 52-54, wherein said method is performed in real-time.

69. A device for measuring a parameter of a tissue, the device comprising:
a) a hollow tube;
b) a distal end of elastic material extending from said hollow tube;
c) a tissue adhered to an exterior surface of said distal end.

70. The device of claim 69 wherein said distal end is approximately spherical.

71. The device of claim 69 wherein said tube communicates with a pressure transducer.

72. The device of claim 71 wherein a change in pressure inside said tube is detected by said pressure transducer.

73. The device of claim 69 wherein said tissue is grown on said exterior surface of said distal end.

74. The device of claim 69 wherein said tissue comprises muscle tissue.

75. The device of claim 74 wherein said muscle tissue comprises cardiac muscle, smooth muscle or striated muscle.

76. The device of claim 74 wherein contraction of said muscle tissue results in a detectable change in pressure inside said tube.

77. The device of claim 69 wherein said elastic material comprises a silicon membrane.

78. A method of determining the bioactivity of a compound, the method comprising contacting a device of claim 69 with said compound and detecting a change in pressure inside said tube.

79. The method of claim 78 wherein said tissue comprises muscle.

80. An array of devices of claim 69.

81. A device comprising:
a) a hollow tube; and
b) an elastic membrane covering a distal end of said tube, said membrane in contact with a tissue.

82. The device of claim 81 wherein said tube communicates with a pressure transducer.

83. The device of claim 81 wherein said membrane comprises a silicon membrane.

84. The device of claim 81 wherein said tissue is formed on said membrane.

85. The device of claim 81 wherein said tissue is not formed on said membrane.

86. The device of claim 81 wherein said tissue comprises muscle tissue.

87. The device of claim 86 wherein said muscle tissue comprises cardiac muscle, smooth muscle or striated muscle.

88. The device of claim 86 wherein contraction of said muscle tissue results in a detectable change in pressure inside said tube.

89. A method of determining the bioactivity of a compound, the method comprising contacting a device of claim 86 with said compound and detecting a change in pressure inside said tube.

90. The method of claim 89 wherein said tissue comprises muscle.

91. An array of devices of claim 81.
